(19) United States
(12) Patent Application Publication
(10) Pub. No.: US 2009/0233982 A1
(43) Pub. Date: Sep. 17, 2009

(54) Methyltransferases and Their Uses

(76) Inventors: Michaela Frye, London (GB); Fiona Watt, Cambridge (GB)

Correspondence Address: FOLEY & LARDNER LLP, P.O. BOX 80278, SAN DIEGO, CA, 92138-0278, US

(21) Appl. No.: 11/887,845

(22) PCT Filed: Mar. 3, 2006
(86) PCT No.: PCT/GB2006/000796

§ 371 Date: Feb. 23, 2009

Related U.S. Application Data
(60) Provisional application No. 60/658,535, filed on Mar. 4, 2005.

(51) Int. Cl. A61K 31/7088; C12N 9/10; C12Q 1/68; C12Q 1/48; G01N 33/53; C07K 16/00; C07H 21/02

(52) U.S. Cl. 514/44 A; 435/193; 435/6; 435/15; 435/7.1; 530/387.9; 536/24.5; 514/44 R

(57) ABSTRACT

The identification and cloning of Misu polypeptide are disclosed and in particular full length human and murine Misu polypeptides. The Misu polypeptides are shown to have methyltransferase activity for a range of nucleic acid substrates. The uses of these polypeptides and nucleic acid molecules are disclosed, for example for screening for compounds that are capable of modulating a Misu biological activity, and the use of Misu inhibitors for the treatment of cancer.

### FIELD OF THE INVENTION

The present invention relates to methyltransferases and their uses, and in particular to the identification, isolation and characterisation of Misu (Myc Induced SUn domain containing protein), a Myc regulated methyltransferase and to uses of these polypeptides, e.g. for screening for modulators of Misu.

### BACKGROUND OF THE INVENTION

Myc was first described as an oncogene when it was found to be translocated in Burkitt's lymphoma over two decades ago (Dalla-Favera et al. 1982; Taub et al. 1982). Since then, it has been reported that nearly all types of human cancer over-express myc (Nesbit et al. 1999). Myc is a nuclear protein that heterodimerizes with the ubiquitously expressed protein Max to function as a sequence specific transcription factor, binding to E box elements (Blackwood and Eisenman 1991; Prendergast et al. 1991; Blackwood et al. 1992a; Blackwood et al. 1992b; Blackwell et al. 1993). Myc/Max heterodimers activate gene transcription by recruiting a protein complex containing histone acetyl transferase (HAT; Eisenman 2001; Frank et al. 2001). Myc and Max repress transcription by interfering with the transcriptional activator Miz-1 or recruiting a co-repressor containing the histone deacetylase complex (HDAC; Satou et al. 2001; Staller et al. 2001; Wanzel et al. 2003).

Overexpression of Myc promotes tumorigenesis, but its cellular functions in normal cells are still enigmatic. Recent studies have revealed a role in regulating adult stem cell homeostasis in the epidermis (Gandarillas and Watt, 1997; Arnold and Watt, 2001; Waikel et al., 2001), intestinal epithelium (van de Wetering et al., 2002) and blood (Wilson et al., 2004). In the intestine, Wnt signaling controls stem cell renewal and Myc is upregulated in response to β-catenin (van de Wetering et al., 2002; Pinto et al. 2003). In the epidermis and haemopoietic tissue, Myc induces exit from the stem cell niche by down-regulating genes encoding proteins involved in cell adhesion, including integrins, extracellular matrix molecules and components of the actin cytoskeleton (Gandarillas and Watt, 1997; Waikel et al., 2001; Frye et al., 2003; Wilson et al. 2004). In addition, Myc impairs epidermal cell migration and this may explain why differentiation into sebocytes and interfollicular epidermis is promoted at the expense of the hair lineages (Waikel et al., 2001; Frye et al., 2003).

Myc overexpression confers a proliferative advantage to tumour cells in both cell culture and animal models (Land et al. 1983; Adams et al. 1985). Studies in Drosophila have demonstrated that elevated levels of Myc provide cells with a competitive advantage, allowing them to proliferate faster and leading to the death of neighbouring cells (de la Cova et al. 2004; Moreno and Basler 2004). Many Myc targets are ribosomal genes; while they could mediate Myc induced cell growth, it is not clear what role they play in Myc induced cell cycle progression (Patel, 2004).

WO 2004/031364 (Incyte Corporation) discloses a nucleic acid sequence number 44, one of a large number of sequences purported to encode polypeptides associated with cell growth, differentiation and death. The application does not produce or characterise in any way the polypeptide encoded by this nucleic acid sequence and sequence 44 is only said to be differentially expressed in association with immune responses. No role of the nucleic acid or polypeptide in connection with any specific disease or conditions is disclosed or suggested.

### SUMMARY OF THE INVENTION

Broadly, the present invention relates to a gene activated by Myc that encodes a previously uncharacterised nucleolar methyltransferase referred to herein as Misu. Evidence is provided to show that transient activation of Myc can lead to epigenetic changes in adult epidermis and that these could involve Misu. When Misu expression is inhibited, Myc induced proliferation is blocked. Since Misu is expressed at low levels in most normal tissues and is overexpressed in different types of tumour, the present invention discloses that Misu is a therapeutic target, in particular for the treatment of hyperproliferative disorders such as cancer. The present invention discloses full length human and murine Misu nucleic acid and polypeptide sequences and their uses, e.g. for screening for compounds that are capable of modulating, and particularly binding to Misu polypeptide or inhibiting a Misu biological activity. The present invention also concerns the treatment and diagnosis of conditions linked to Misu expression, in particular hyperproliferative diseases such as cancer. These applications may be by virtue of Misu being linked to Myc functions in controlling cell proliferation, but also as a target for anti-cancer therapies in its own right.

Accordingly, in a first aspect, the present invention provides an isolated Misu polypeptide comprising the amino acid sequence set out in SEQ ID NO:2 or 4. SEQ ID NO:1 and 2 show the cDNA and predicted amino acid sequence respectively of wild-type mouse Misu, a 2271 nucleotide cDNA sequence that encodes a 757 amino acid polypeptide. SEQ ID NO: 3 and 4 show the cDNA and predicted amino acid sequence respectively of wild-type human Misu, a 2301 nucleotide cDNA sequence that encodes a 767 amino acid polypeptide. Human and murine Misu share 87.2% amino acid sequence identity and 82.8% nucleic acid sequence identity. SEQ ID NO: 5 shows the human Misu nucleic acid sequence including flanking sequences.

The present invention may further employ an isolated Misu polypeptide having at least 80% amino acid sequence identity with the amino acid sequence set out in SEQ ID NO: 2 or 4, e.g. in assays, methods and uses disclosed herein. Preferred embodiments of the invention provide isolated polypeptides having at least 90% sequence identity with the sequence set out in SEQ ID NO: 2 or 4.

In a further aspect, the present invention provides an isolated polypeptide which has at least 80% amino acid sequence identity with amino acids 1 to 236 or 237 to 767 of SEQ ID NO: 2 or 4, wherein the polypeptide is capable of inhibiting methyltransferase activity of the polypeptide having the amino acid sequence set out in SEQ ID NO: 2 or 4.

In a further aspect, the present invention provides an isolated polypeptide which is encoded by a nucleic acid sequence which is capable of hybridising under stringent conditions to the nucleic acid sequence set out in SEQ ID NO:1 or 3, or a complementary sequence thereof, e.g. in assays, methods and uses disclosed herein.

As defined below, preferred polypeptides employed in accordance with the present invention possess a Misu biological activity, and especially activity as a methyltransferase. This is discussed further below.

In a further aspect, the present invention provides a substance which is a fragment, active portion or sequence variant of one of the above polypeptides. Preferred fragments and active portions comprise all or part of the Misu amino acid sequence set out in SEQ ID NO: 2 and 4.

In a further aspect, the present invention provides a Misu polypeptide as defined above joined to a coupling partner.

In a further aspect, the present invention provides an isolated nucleic acid molecule encoding one of the above polypeptides, and complementary nucleic acid sequences thereof. The cDNA sequence of full length mouse and human Misu is shown in SEQ ID NO: 1 and 3. The present invention also includes nucleic acid molecules having greater than a 90% sequence identity with one of the above nucleic acid sequence. In other embodiments, the present invention relates to nucleic acid sequences which hybridise to the coding sequence set out in SEQ ID NO: 1 and 3, or a complementary sequence thereof.

In further aspects, the present invention provides an expression vector comprising one of the above nucleic acid operably linked to control sequences to direct its expression, and host cells transformed with the vectors. The present invention also includes a method of producing a Misu polypeptide, or a fragment or active portion thereof, comprising culturing the host cells and isolating the polypeptide thus produced. Also included is a method of producing a Misu polypeptide by in vitro translation from the expression vector.

In a further aspect, the present invention provides a composition comprising one or more of the above polypeptides or nucleic acid molecules as defined herein.

In a further aspect, the present invention provides the use of a Misu polypeptide as defined herein (including fragments, active portions or sequence variants), or a corresponding nucleic acid molecule, for screening for candidate compounds which (a) share a Misu biological activity or (b) bind to the Misu Polypeptide or (c) inhibit a biological activity of a Misu polypeptide or (d) inhibit the expression of Misu polypeptide. By way of example, screening can be carried out to find substances which can modulate Misu polypeptides or inhibit Misu expression to develop as lead compounds in pharmaceutical research.

Thus, in a further aspect, the present invention provides a method of identifying a compound which is capable of modulating an activity of a Misu polypeptide, the method comprising:

(a) contacting at least one candidate compound with a Misu polypeptide as defined herein under conditions in which the candidate compound and Misu polypeptide are capable of interacting;

(b) determining in an assay for a Misu activity whether the candidate compound modulates the activity; and,

(c) selecting a candidate compound which modulates an activity of the Misu polypeptide.

Thus, in a further aspect, the present invention provides a method of identifying a compound which is capable of binding to a Misu polypeptide, the method comprising:

(a) contacting at least one candidate compound with a Misu polypeptide as defined herein under conditions in which the candidate compound and Misu polypeptide are capable of interacting;

(b) determining in an assay for a Misu activity whether the candidate compound binds to the Misu polypeptide; and,

(c) selecting a candidate compound which binds to the Misu polypeptide.

In a further aspect, the present invention provides a method of identifying a compound which is capable of inhibiting Misu polypeptide, the method comprising:

(a) contacting at least one candidate compound and a Misu polypeptide as defined herein in the presence of a substrate for Misu under conditions in which the candidate compound, Misu polypeptide and Misu substrate are capable of interacting;

(b) determining whether the candidate compound inhibits the activity of the Misu polypeptide in reacting with the substrate; and,

(c) selecting a candidate compound which inhibits the activity of the Misu polypeptide on the substrate.

In aspects of the present invention relating to screening methods, a substrate of Misu polypeptide may be employed to determine whether a candidate compound or substance is capable of inhibiting an enzymatic activity of the Misu polypeptide, e.g. as a methyltransferase.

Conveniently, the substrate is a nucleic acid molecule, most conveniently an oligonucleotide, that is capable of being methylated by Misu. Preferred substrates include ribonucleic acid molecules such as mRNA, tRNA, rRNA or miRNA. In one embodiment, the effect of a candidate compound in modulating the activity of Misu can be assessed using a labelled nucleic acid substrate comprising a restriction site that is removed when Misu methylates the substrate, thereby inhibiting the cleavage of the restriction site by the restriction enzyme. Conveniently, the label of the substrate is a fluorescent label. However, other methods of determining the inhibition of Misu by a candidate compound will be apparent to the skilled person, for example employing antibodies which are capable of specifically binding to unmethylated or methylated substrates. As discussed below, the method described herein are useful for screening for modulators of Misu which may be useful in the treatment of a hyperproliferative disorder such as cancer. Examples of high-throughput screening assays that measure DNA methylation and that can be adapted for use in accordance with the present invention is provided in Eads et al, Nucleic Acid Research, 28(8): 2000, e32i-viii.

For example, in one embodiment, a nucleic acid having a fluorophore and a quencher and having a restriction site which is not cleaved when methylated situated between the fluorophore and quencher may be used. When the nucleic acid is cleaved by a restriction enzyme, the fluorophore is released from the quencher, which can be detected as an increase in fluorescence. Methylation of the nucleic acid by Misu may thus be detected as a reduction of the increase in fluorescence which occurs when the nucleic acid is incubated with a restriction enzyme. Alternatively, a nuclease site other than a restriction enzyme site may be used, or different permutations of fluorophors and quenchers to produce different signal readouts in the assay.

In another embodiment, methylation of a nucleic acid substrate is detected by treatment with sodium bisulphite, which alters the sequence of methylated nucleic acids by changing unmethylated cytosines to uracil but leaving methylated cytosines unaffected. The change in sequence may be detected by sequencing, or by PCR using primers that hybridise with only one of the potential sequences. See, for example, Herman et al, P.N.A.S. USA, 93: 9821-9826, 1996.

In a further embodiment, the nucleic acid substrate having a quencher and fluorophore and a restriction site may be replaced with a substrate having a fluorescent label that is used with a binding agent (e.g. a protein) that specifically binds to a methylation site on the substrate when the substrate is methylated, and which also has a fluorescent label. Methylation of the substrate could then be detected in a fluorescent polarisation assay. Thus, if methylation occurs, the binding agent would bind to the substrate and the proximity of the fluorescent labels would lead to a change in fluorescent signal compared to the label on the substrate alone. These changes in fluorescence may therefore be used to determine whether candidate compounds are capable of inhibiting the methyltransferase activity of Misu.

A further aspect of the invention provides a modulator (e.g. an inhibitor) of Misu polypeptide activity identified or identifiable by the methods described herein

In another aspect, the present invention provides a method of identifying compounds that inhibit or increase the expression of Misu. Such compounds may be conveniently identified in a cell-based assay and Misu expression detected by RT-PCR or antibody detection.

In one embodiment, the present invention provides a method of identifying a compound which is a modulator of Misu mRNA expression, the method comprising:

(a) introducing a candidate compound into cells expressing Misu mRNA

(b) determining whether Misu mRNA expression is altered in the presence of the candidate compound

(c) selecting a candidate compound which modulates the expression of the Misu mRNA.

In another embodiment, the present invention provides a method of identifying a compound which is a modulator of Misu polypeptide expression, the method comprising:

(a) introducing a candidate compound into a cell expressing Misu polypeptide or mRNA;

(b) determining whether the Misu polypeptide or mRNA expression is altered in the presence of the candidate compound;

(c) selecting a candidate compound which modulates the expression of the Misu polypeptide or mRNA.

In certain embodiments, the cell expressing Misu mRNA and/or protein expresses a Myc-ER fusion protein as described herein, in order to stimulate Misu expression. Conveniently, the cell is a keratinocyte, and more preferably a human keratinocyte. The culturing of these cells is described in Watt et al, Cell Biology: A Laboratory Handbook, p 83-89, Academic Press, Inc, 1994.

More generally, any of the screening methods described herein may be carried out in a cell based format in cells, such as keratinocytes, where one or more candidate compounds are added to cells expressing Misu to determine whether the compounds are capable of modulating Misu activity or expression. This may be carried out as described in the experimental section below. In one embodiment, the cells express Misu mRNA and/or protein and/or express a Myc-ER fusion protein as described herein, in order to stimulate Misu expression, and then can be used to screen candidate compounds, e.g. as Misu inhibitors.

A further aspect of the invention provides a modulator of Misu expression identified or identifiable by the methods described herein. Such compounds may, for example, interfere with the ability of Myc to bind to the Misu promoter, or may bind to the Misu promoter directly. Other modulators of Misu expression include Misu antisense RNAs and siRNAs, which are discussed in more detail below.

In a further aspect, the present invention provides antibodies capable of specifically binding to the above Misu polypeptides, or an active portion, domain or fragment thereof, and the use of the Misu polypeptide or peptides based on the sequence for designing antibodies or for use in a method of preparing antibodies capable of binding to Misu. These antibodies can be used in assays to detect and quantify the presence of Misu polypeptide, in methods of purifying Misu polypeptides, and as modulators, for example inhibitors, of Misu polypeptides.

In a further aspect, the present invention provides a method of amplifying a nucleic acid test sample comprising priming a nucleic acid polymerase reaction with nucleic acid encoding a Misu polypeptide as defined above.

In a further aspect, the present invention provides a method for the diagnosis or prognosis of hyperproliferative diseases such as cancer, the method comprising determining the presence or amount of Misu polypeptide, or an isoform or splice variant thereof, or the presence or amount of Misu nucleic acid, in a sample from a patient. This is discussed in more detail below.

The work described herein shows that Misu is overexpressed in cancer, and in particular in breast cancer, colon cancer and skin cancer, and Crohn's disease. Other conditions in which Misu may be implicated are conditions associated with aberrant Myc activity as Misu is demonstrated herein to control Myc role in causing cell proliferation. Aberrant Myc activity has been associated with various forms of cancer including Burkitt's lymphoma, diffuse large B-cell lymphomas, breast cancer, prostate cancer, gastrointestinal cancer including human colon adenocarcinoma, colorectal cancer, gastric cancer, multiple myeloma, melanoma, myeloid leukaemia and lymphoma, neuroblastoma, small cell lung cancer, medullary thyroid carcinoma, retinoblastoma, alvelolar rhabdomyosarcoma, breast cancer, uterine cancer and bowel cancer. Misu may also play a role in other conditions, including tumour angiogenesis, chronic inflammatory disease such as rheumatoid arthritis, atherosclerosis, skin diseases such as psoriasis, eczema, and scleroderma, diabetes, diabetic retinopathy, retinopathy of prematurity and age-related macular degeneration.

Accordingly, in a further aspect, the present invention provides the use of an inhibitor of a Misu polypeptide, a Misu polypeptide that is capable of inhibiting the methyltransferase activity of the polypeptide of SEQ ID NO: 2 or 4 or a substance which inhibits Misu expression, for the preparation of a medicament for the treatment of these conditions.

Methods of treatment of hyperproliferative diseases using an inhibitor of Misu activity of expression are also provided.

These and other aspects of the present invention are described in more detail below. By way of example and not limitation, embodiments of the present invention will now be described in more detail with reference to the accompanying figures.

### BRIEF DESCRIPTION OF THE FIGURES

FIG. 1. Expression of Misu RNA correlates with Myc activation in vitro and in vivo. (A) RNA expression profile of Misu in skin of K14MycER transgenic and wild type mice that were untreated (0 d) or treated with 4OHT for one day or four days (1 d and 4 d). (B) Northern Blot of total RNA from skin of wild-type (wt) and transgenic (tg) mice, treated for four days (4 d) or nine days (9 d) with 4OHT. Probed for Misu (arrow) or, as a loading control, 18S RNA. (C) Sequence surrounding the first conserved cysteine within the SUN domain of Misu, DNA (HhaI, EcoRI, mDnmt1) and RNA MTases (NCL1, p120, Nop2). (D) Tissue Northern blot probed for Misu (arrow). (E-L) In situ hybridisation of Misu in back skin of wild-type mouse treated with 4OHT for 4 days (E,F) or K14MycER transgenic untreated (G,H) or treated with 4OHT for 1 day (I,J) or 4 days (K,L). Bright and dark field views of the same sections are shown. Scale bar: 100 μm

FIG. 2. Genomic organisation of Misu and detection of splice variants. (A) Mouse Misu. Exon A represents a putative splice variant not found in human. (B) Human Misu. In both species exon 4 can be deleted by alternatively splicing exon 3 to exon 5. (C) Schematic overview of the putative splice variants of Misu showing the full length protein of 85 kDa, the 23 kDa protein (red asterisk) generated when exon 4 is deleted, and a 60 kDa form (blue asterisk) generated when the gene is transcribed from the start methionine located in exon 6. The location of peptides used to generate antibodies EF1 and EF2 is shown. (D) RT-PCR. Upper panel and left hand lanes of lower panel: primers detected full length Misu, containing exon 4 (upper band) and the splice variant encoding the 23 kD protein generated when exon 4 is deleted (red asterisk). Human cells examined were A431, EJ28, JAR, primary human keratinocytes (k) and dermal fibroblasts (HDF). A PCR reaction without template was the negative control and amplification of GAPDH served as a standard. In mouse skin (m-skin) and embryonic stem cells (m-ES), the variant resulting from splicing exon A to exon 1 was not amplified (lower panel, right hand lanes). M: molecular weight markers, (E) EF1 and EF2 detected the full length Misu protein at 97 kDa in human keratinocytes (k). EF1 detected an additional band of around 30 kDa (red asterisk) and EF2 detected additional bands of 60 kDa (ΔMisu; blue asterisk, also in C) and 45 kDa (nonspecific). (F) Western Blot showing up-regulation of Misu by activation of MycER in human keratinocytes (k) after 2 (2 d) or 3 days (3 d) of 4OHT treatment, compared to controls (k-pBabe; k-106ER). Actin served as a loading control.

FIG. 3. Expression of Misu in human and mouse skin. Histology (A,E,I,C,G) and Misu immunofluorescence staining with MFRY5 (B,D,F,H,J) of wild-type mouse back skin (A,B,E,F,I,J) and K14MycER back skin following 4OHT treatment (C,D,G,H). Arrowheads mark cells with high Misu expression. (K,L) Immunohistochemistry (IHC) of human skin, showing bulb of anagen hair follicle (K) and sebaceous gland (L). IFE: interfollicular epidermis; SG: sebaceous gland; HF: hair follicle. Scale bars: 50 μm

FIG. 4. Misu methylates RNA and DNA targets in vitro and localises to the nucleoli, centrioles, and the mitotic spindle of cells, according to cell cycle status. (A) Incorporation of radioactive (¹⁴C) labelled SAM into non-(noCH3) or hemi-(hemiCH3) methylated DNA, rRNA, or tRNA mediated by in vitro translated Misu or ΔMisu compared to the DNA MTases Dnmt1 or SssI as positive controls. As negative controls Misu and Dnmt1 (Control) were transcribed in the anti-sense direction. Right panel: lysates were treated with RNase or DNase prior scintillation counting. (B-M) Immunofluorescence staining for Misu (green) and nucleolin (red in B-G), mitochondrial dye (Mito, red in H-J), γ-tubulin (γTub, red in K-M) with DAPI counterstaining (blue). D, G, J, M are merged images of B and C, E and F, H and I, K and L, respectively. SZ95 were in early G1 phase (B-D), G1 and S (E-C), G2 (H-J) or mitosis (K-M). B-D: Arrow shows co-localisation of Misu with nucleolin at nucleoli. E-G: Cells in G1 (arrow) and S phase (asterisks) are shown. H-J: cytoplasmic Misu does not colocalise with mitochondria (arrow). K-M: colocalisation of Misu with γ-tubulin at spindle poles. Scale bars: 10 μm

FIG. 5. RNA dependent co-localisation of Misu with histone 3.3 in nucleoli. (A-L) Immunofluorescence staining of Misu (A-D), nucleolin (red in E-H) and H3.3 (1-L) in SZ95 cells treated with PBS (A,E,I), RNase (5 min: B,F,J; 20 min: C,G,K) or DNase (15 min, D,H,L). M,N: Immunofluorescence staining of H3.3 in 4OHT treated SZ95 cells transduced with MycER (N) or 106ER (M). (O,P) H3.3 expression in interfollicular epidermis of wild-type (O) and 4OHT treated K14MycER transgenic mice. Scale bars: 5 μm (Q,R) Whole mount labelling of 4OHT treated wild-type (Q) or K14MycER(R) tail epidermis with an antibody to H3.3. Locations of sebaceous glands (SG), hair follicle outer root sheath (HF) and bulb of anagen follicles are shown. DAPI nuclear counterstain is shown in blue in panels E-H and M-R. Scale bars: 100 μm

FIG. 6. Misu mediates Myc induced keratinocyte proliferation and differentiation. (A-D) RNAi knockdown of Misu expression by retroviral infection of primary human keratinocytes (top panels) and transient transfection of SZ95 cells (lower panels). Immunofluorescence staining shows reduced nuclear staining for misu (green) in cells expressing Misu RNAi (C) compared to cells expressing empty vector (A). B,D: DAPI nuclear counterstain of cells in A,C, respectively. Arrowheads in C and D show cells with reduced nuclear Misu. (G) The effect of Misu RNAi was confirmed by Western blotting of keratinocytes transduced with Misu RNAi alone or in combination with MycER. E,F,H: Introduction of the Misu RNAi construct into keratinocytes transduced with MycER (k-MycER) or empty retroviral vector (k-pRS, k-pBabe) led to inhibition of proliferation. E,F: representative dishes; H: growth curve. I-T: Histology (I-L) and immunofluorescence staining (M-T) of keratinocytes cultured on DEDs. Keratinocytes were infected with empty vector (k-ev; I,M,Q), MisuRNAi (J,N,R), MycER (K,O,S) or both MisuRNAi and MycER (L,P,T) and grown in the presence of 4OHT. M-P: stained with antibody to transglutaminase 1; Q-T: stained with anti-Ki67. M-T: counterstained with DAPI (blue). Scale bars: 100 μm

FIG. 7. Expression of Misu normal tissues and in tumours. A-I: Immunohistochemistry for Misu in normal human lymph node (A), liver (B) and pancreas (C), primary breast carcinoma (BC) (E), lymph node metastasis of breast carcinoma (F), normal colonic epithelium (G), colon carcinoma (H) and Crohn's disease (I). D: Control staining of human breast with anti-Misu omitted. K-M: immunofluorescence staining of Misu in mouse skin papilloma (K) and squamous cell carcinoma. Adjacent sections (J,L) were stained with H & E. Arrows show Misu positive cells. Scale bar: 100 μm.

FIG. 8. An alignment of the mouse and human Misu sequences, marking the putative locations of the SUN domain and active site and showing two splice variants.

FIG. 9. (A) Western Blot and (B) Immunofluorescence of dose dependent RNAi knock-down of Misu by using different RNAi constructs (RNAi1, 3 and 4).

FIG. 10. Infection of Misu RNAi constructs into the squamous cell carcinoma cell line SCC15. Infected cell were subcutaneously injected into Nude mice and tumours were analysed after 4 weeks. (A) Two mice with two infection sites each were analysed and the number of developed tumours counted. (B) Misu RNAi constructs decrease tumour size in a dose dependent manner. (C) Injection of SCC15 cells infected with a control plasmid (pRS) result in development of squamous cell carcinomas (left hand panels). Histology of tumours derived from SCC15 cells infected with RNAi3 or RNAi4 (middle and right hand panels) differ from controls and resemble a cyst with high accumulation of cornified material (arrows).

### MISU NUCLEIC ACID

A “Misu nucleic acid” includes a nucleic acid molecule which has a nucleotide sequence encoding a polypeptide comprising the amino acid sequence shown in SEQ ID NO: 2 or 4, or any one of the other Misu polypeptides of the present invention. The Misu coding sequence may be the full length nucleic acid sequence shown in SEQ ID NO: 1 or 3, a complementary nucleic acid sequence, or it may be a sequence variant differing from one of the above sequences by one or more of addition, insertion, deletion and substitution of one or more nucleotides of the sequence shown. Changes to a nucleotide sequence may result in an amino acid change at the protein level, or not, as determined by the genetic code. Nucleic acid encoding a polypeptide which is a sequence variant preferably has at least 80% sequence identity, more preferably at least 95% sequence identity, more preferably at least 98% sequence identity, and most preferably at least 99% sequence identity with the nucleic acid sequence shown in SEQ ID NO: 1 or 3.

The present invention also includes fragments of the Misu nucleic acid sequences described herein, the fragments preferably being at least 60, 120, 180, 240, 480 or 960 nucleotides in length. Generally, nucleic acid according to the present invention is provided as an isolate, in isolated and/or purified form, or free or substantially free of material with which it is naturally associated. Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

The present invention also includes nucleic acid molecules which are capable of hybridising to one of the Misu sequences disclosed herein, or a complementary sequence thereof. Stringency of hybridisation reactions is readily determinable by one of ordinary skill in the art, and generally is an empirical calculation dependent upon probe length, washing temperature and salt concentration. In general, longer probes require higher temperatures for proper annealing, while shorter probes need lower temperatures. Hybridisation generally depends on the ability of denatured DNA to re-anneal when complementary strands are present in an environment below their melting temperature. The higher the degree of desired homology between the probe and hybridisable sequence, the higher the relative temperature which can be used. As a result, it follows that higher relative temperatures would tend to make the reaction conditions more stringent, while lower temperatures less so. For additional details and explanation of stringency of hybridisation reactions, see Ausubel et al, Current Protocols in Molecular Biology, Wiley Interscience Publishers, (1995).

Preferably, a nucleic acid sequence will hybridise to a Misu sequence of the invention, or a complementary sequence thereof under “stringent conditions”. These are well known to those skilled in the art and include those that: (1) employ low ionic strength and high temperature for washing, for example 0.015 M sodium chloride/0.0015 M sodium citrate/0.1% sodium dodecyl sulfate at 50° C.; (2) employ during hybridisation a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 760 mM sodium chloride, 75 mM sodium citrate at 42° C.; or (3) employ 50% formamide, 5×SSC (0.75 M NaCl, 0.075 M sodium citrate), 50 mM sodium phosphate (pH 6 8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

Nucleic acid sequences encoding all or part of the Misu gene and/or its regulatory elements can be readily prepared by the skilled person using the information and references contained herein and techniques known in the art (for example, see Sambrook, Fritsch and Maniatis, Molecular Cloning, A Laboratory Manual, Cold Spring Harbour Laboratory Press, 1989, and Ausubel et al, Short Protocols in Molecular Biology, John Wiley and Sons, 1992). These techniques include (i) the use of the polymerase chain reaction (PCR) to amplify samples of such nucleic acid, e.g. from genomic sources, (ii) chemical synthesis, or (iii) amplification in E. coli. Modifications to the Misu sequences can be made, e.g. using site directed mutagenesis, to provide expression of modified Misu polypeptide or to take account of codon preference in the host cells used to express the nucleic acid.

PCR techniques for the amplification of nucleic acid are described in U.S. Pat. No. 4,683,195. The Misu nucleic acid sequences provided herein readily allow the skilled person to design PCR primers. References for the general use of PCR techniques include Mullis et al, Cold Spring Harbour Symp. Quant. Biol., 51:263, (1987), Ehrlich (ed), PCR Technology, Stockton Press, NY, 1989, Ehrlich et al, Science, 252:1643-1650, (1991), “PCR protocols; A Guide to Methods and Applications”, Eds. Innis et al, Academic Press, New York, (1990).

In order to obtain expression of the Misu nucleic acid sequences, the sequences can be incorporated in a vector having control sequences operably linked to the Misu nucleic acid to control its expression. The vectors may include other sequences such as promoters or enhancers to drive the expression of the inserted nucleic acid, nucleic acid sequences so that the Misu polypeptide is produced as a fusion and/or nucleic acid encoding secretion signals so that the polypeptide produced in the host cell is secreted from the cell. Suitable vectors can be chosen or constructed, containing appropriate regulatory sequences, including promoter sequences, terminator fragments, polyadenylation sequences, enhancer sequences, marker genes and other sequences as appropriate. Vectors may be plasmids or viral, e.g. phage, or phagemid, as appropriate. For further details see, for example, Molecular Cloning: a Laboratory Manual: 2nd edition, Sambrook et al., 1989, Cold Spring Harbour Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992. Misu polypeptides can be obtained by transforming the vectors into host cells in which the vector is functional, culturing the host cells so that the Misu polypeptide is produced and recovering the Misu polypeptide from the host cells or the surrounding medium. Prokaryotic and eukaryotic cells are used for this purpose in the art, including strains of E. coli, insect cells (e.g. transformed with baculovirus), yeast, and eukaryotic cells such as COS or CHO cells. The choice of host cell can be used to control the properties of the Misu polypeptide expressed in those cells, e.g. controlling where the polypeptide is deposited in the host cells or affecting properties such as its glycosylation and phosphorylation. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components such as a carrier. Polypeptides may also be expressed in in vitro systems, such as reticulocyte lysate.

The nucleic acid sequences provided herein are useful for identifying Misu nucleic acid in a test sample. The present invention provides a method which includes hybridising a probe sharing all or part of the sequence provided herein, or a complementary sequence, to the target nucleic acid. Hybridisation is generally followed by identification of successful hybridisation and isolation of nucleic acid which has hybridised to the probe, which may involve one or more steps of PCR. These methods may be useful in determining whether Misu nucleic acid is present in a sample, e.g. in a particular type of cells present in the sample.

Nucleic acid according to the present invention is obtainable using one or more oligonucleotide probes or primers designed to hybridise with one or more fragments of the nucleic acid sequence shown herein, particularly fragments of relatively rare sequence, based on codon usage or statistical analysis. A primer designed to hybridise with a fragment of the nucleic acid sequence shown in the above figures may be used in conjunction with one or more oligonucleotides designed to hybridise to a sequence in a cloning vector within which target nucleic acid has been cloned, or in so-called “RACE” (rapid amplification of cDNA ends) in which cDNA's in a library are ligated to an oligonucleotide linker and PCR is performed using a primer which hybridises with the sequence shown herein and a primer which hybridises to the oligonucleotide linker.

On the basis of amino acid sequence information, oligonucleotide probes or primers may be designed, taking into account the degeneracy of the genetic code, and where appropriate, codon usage of the organism from the candidate nucleic acid is derived. An oligonucleotide for use in nucleic acid amplification may have about 10 or fewer codons (e.g. 6, 7 or 8), i.e. be about 30 or fewer nucleotides in length (e.g. 18, 21 or 24). Generally specific primers are upwards of 14 nucleotides in length, but not more than 18-20. Those skilled in the art are well versed in the design of primers for use processes such as PCR.

Accordingly, a further aspect of the present invention provides an oligonucleotide or nucleotide fragment of the one of the nucleotide sequence disclosed herein, or a complementary sequence, in particular for use in a method of obtaining and/or screening nucleic acid. The sequences referred to above may be modified by addition, substitution, insertion or deletion of one or more nucleotides, but preferably without abolition of ability to hybridise selectively with nucleic acid with the sequence shown herein, that is wherein the degree of sequence identity of the oligonucleotide or polynucleotide with one of the sequences given is sufficiently high.

In some preferred embodiments, oligonucleotides according to the present invention that are fragments of any of the nucleic acid sequences provided herein, or complementary sequences thereof, are at least about 10 nucleotides in length, more preferably at least about 15 nucleotides in length, more preferably at least about 20 nucleotides in length. Such fragments themselves individually represent aspects of the present invention. Fragments and other oligonucleotides may be used as primers or probes as discussed but may also be generated (e.g. by PCR) in methods concerned with determining the presence of Misu nucleic acid in a test sample.

### Misu Polypeptides

The Misu polypeptides disclosed herein, or fragments or active portions thereof, can be used as pharmaceuticals, in the developments of drugs, for further study into its properties and role in vivo and to screen for Misu inhibitors. Thus, a further aspect of the present invention provides a polypeptide which has the amino acid sequence shown in SEQ ID NO: 2 or 4, which may be in isolated and/or purified form, free or substantially free of material with which it is naturally associated, such as polypeptides other than Misu.

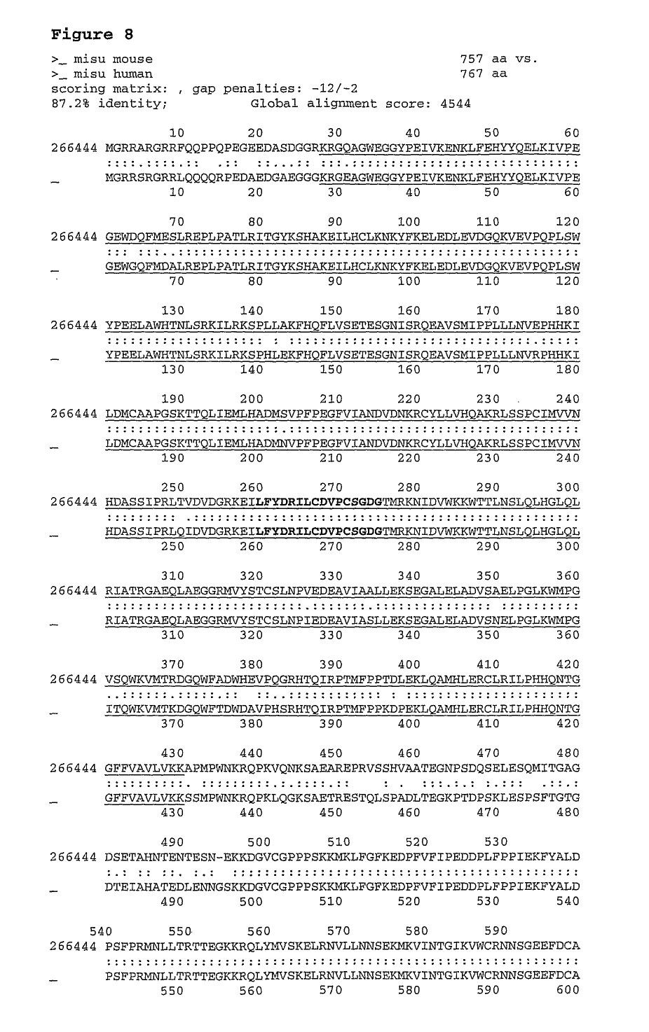

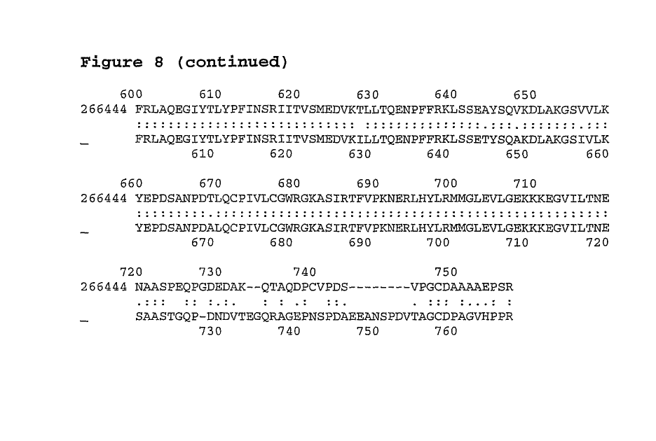

FIG. 8 shows an alignment of the human and murine amino acid sequences of Misu. The SUN domain is located between amino acids 28 and 430 inclusive of both human and murine Misu (shown underlined) and the active site is located between amino acids 260 and 275 inclusive (shown in bold type). Two splice variants have been found one consisting of the N-terminal portion of Misu up to and including the isoleucine residue at position 236 and the other consisting of the C-terminal portion starting from the methionine residue at position 237. The first splice variant lacks the active site, while the second splice variant include the active site, but only part of the SUN domain and is dominant negative.

The present invention also includes active portions, domains and fragments (including domains) of the Misu polypeptides of the invention.

An “active portion” of Misu polypeptide means a peptide which is less than said full length Misu polypeptide, but which retains at least some of its essential biological activity, e.g. as a methyltransferase, preferably by including the active site located between residues 260 and 275 of the sequences shown in FIG. 8, optionally in combination with the SUN domain between amino acids 28 and 430. Active portions may be greater than 100 amino acids, more preferably greater than 200 amino acids, more preferably greater than 300 amino acids and most preferably greater than 400 amino acids in length.

A “fragment” of the Misu polypeptide means a stretch of amino acid residues of at least 5 contiguous amino acids from the sequences set out as SEQ ID NO: 2 or 4, or more preferably at least 10 contiguous amino acids, or more preferably at least 20 contiguous amino acids or more preferably at least 50 contiguous amino acids or more preferably at least 100 contiguous amino acids. Fragments of the Misu polypeptide sequences may be useful as antigenic determinants or epitopes for raising antibodies to a portion of the Misu amino acid sequence which also forms part of the present invention. Fragments of Misu can also act as sequestrators or competitive inhibitors by interacting with other proteins, e.g. if they possess a protein interaction domain present in the full length Misu sequence.

Polypeptides which are amino acid sequence variants are also provided by the present invention. A “sequence variant” of the Misu polypeptide, or an active portion or fragment thereof, means a polypeptide modified by varying the amino acid sequence of the protein, e.g. by manipulation of the nucleic acid encoding the protein or by altering the protein itself. Such sequence variants of the natural amino acid sequence may involve insertion, addition, deletion or substitution of one, two, three, five, ten, twenty or more amino acids.

One class of preferred Misu polypeptides as defined herein have a Misu biological activity as a methyltransferase, that is, they are capable of methylating a substrate such as a nucleic acid including DNA and, more preferably RNA, such as mRNA, tRNA, rRNA and miRNA. However, a further useful class of Misu polypeptides are those which have reduced or do not possess a Misu biological activity and which may therefore serve as competitive inhibitors of biologically active Misu, e.g. a Misu polypeptide which is present endogenously and has methyltransferase activity. In this approach, these Misu polypeptides may act as sequesters or compete with biologically active Misu for binding to substrates, other proteins (e.g. cofactors) and therefore reduce the biological effect of Misu. These include Misu polypeptides that lack the SUN domain set out between amino acids K28 and K430 of SEQ ID NO: 2 and 4, or the active site within the SUN domain (residues L260 to G275 of SEQ ID No. 2 and 4), for example the splice variants described in the experimental section below. One splice variant has only amino acids 1 to 236 of SEQ ID No. 2 or 4 and thus lacks the active site. Another lacks amino acids 1-236 and is thus missing part of the SUN domain, although it carries the active site. This splice variant appears to have dominant negative activity despite retaining the active site.

Misu polypeptides include polypeptides which have at least 80% sequence identity, more preferably at least 90% sequence identity, more preferably at least 95% sequence identity, more preferably at least 97% sequence identity more preferably at least 98% sequence identity and most preferably at least 99% sequence identity the amino acid sequence shown in SEQ ID NO: 2 or 4, or one of the splice variants defined above and shown in FIG. 8.

The skilled person can readily make sequence comparisons and determine identity using techniques well known in the art, e.g. using the GCG program which is available from Genetics Computer Group, Oxford Molecular Group, Madison, Wis., USA, Version 9.1. Particular amino acid sequence variants may differ from those shown in SEQ ID Nos: 2 or 4 by insertion, addition, substitution or deletion of 1 amino acid, 2, 3, 4, 5-10, 10-20 20-30, 30-50, 50-100, 100-150, or more than 150 amino acids.

“Percent (%) amino acid sequence identity” with respect to the Misu polypeptide sequences is defined as the percentage of amino acid residues in a candidate sequence that are identical with the amino acid residues in the Misu sequence, after aligning the sequences and introducing gaps, if necessary, to achieve the maximum percent sequence identity, and not considering any conservative substitutions as part of the sequence identity. Sequence alignment can be carried out by the skilled person using techniques well known in the art for example using publicly available software such as BLAST, BLAST2 or Align software, see Altschul et al (Methods in Enzymology, 266:460-480 (1996); http://blast.wustl/edu/blast/README.html) or Pearson et al (Genomics, 46, 24, 36, 1997). The Align program is available from: http://molbiol.soton.ac.uk/compute/align.html and the percentage sequence identities reported herein and in accordance with the present invention use this program with its default settings. More generally, the skilled person can readily determine appropriate parameters for determining alignment, including any algorithms needed to achieve maximal alignment over the full length of the sequences being compared.

By way of further example, WU-BLAST-2 uses several search parameters, most of which are set to the default values. The adjustable parameters are set with the following values: overlap span=1, overlap fraction=0.125, word threshold(T)=11. The HSPS and HSPS2 parameters are dynamic values and are established by the program itself depending upon the composition of the particular sequence and composition of the particular database against which the sequence of interest is being searched; however, the values may be adjusted to increase sensitivity. A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

Similarly, “percent (%) nucleic acid sequence identity” with respect to the coding sequence of the Misu polypeptides identified herein is defined as the percentage of nucleotide residues in a candidate sequence that are identical with the nucleotide residues in the Misu coding sequence as provided in SEQ ID NO: 1 and 3.

A polypeptide according to the present invention may be isolated and/or purified (e.g. using an antibody) for instance after production by expression from encoding nucleic acid. Polypeptides according to the present invention may also be generated wholly or partly by chemical synthesis. The isolated and/or purified polypeptide may be used in formulation of a composition, which may include at least one additional component.

The Misu polypeptides can also be linked to a coupling partner, e.g. an effector molecule, a label, a drug, a toxin and/or a carrier or transport molecule. Techniques for coupling the peptides of the invention to both peptidyl and non-peptidyl coupling partners are well known in the art.

### Antibodies Capable of Binding Misu Polypeptides

A further important use of the Misu polypeptides is in raising antibodies that have the property of specifically binding to the Misu polypeptides or fragments thereof. The techniques for producing monoclonal antibodies to Misu protein are well established in the art. The anti-Misu antibodies of the present invention may be specific in the sense of being able to distinguish between the polypeptide it is able to bind and other human polypeptides for which it has no or substantially no binding affinity (e.g. a binding affinity of about 1000× worse). Specific antibodies bind an epitope on the molecule which is either not present or is not accessible on other molecules. Other preferred antibodies include those capable of neutralising or inhibiting a Misu biological activity and especially biological activity as a methyltransferase. Thus, antibodies are a class of substances that may be employed in the medical uses disclosed herein which involve inhibiting the activity of Misu, especially in the treatment of cancer.

Antibodies may be obtained using techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992). Isolation of antibodies and/or antibody-producing cells from an animal may be accompanied by a step of sacrificing the animal. As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO 92/01047. The library may be naive, that is constructed from sequences obtained from an organism which has not been immunised with any of the proteins (or fragments), or may be one constructed using sequences obtained from an organism which has been exposed to the antigen of interest.

Antibodies according to the present invention may be modified in a number of ways that are well known in the art. Indeed the term “antibody” should be construed as covering any binding substance having a binding domain with the required specificity. Thus the invention covers antibody fragments, derivatives, functional equivalents and homologues of antibodies, including synthetic molecules and molecules whose shape mimics that of an antibody enabling it to bind an antigen or epitope. Humanised antibodies in which CDRs from a non-human source are grafted onto human framework regions, typically with the alteration of some of the framework amino acid residues, to provide antibodies which are less immunogenic than the parent non-human antibodies, are also included within the present invention.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRS), of an antibody to the constant regions, or constant regions plus framework regions, of a different immunoglobulin. See, for instance, EP 0 184 187 A, GB 2 188 638 A or EP 0 239 400 A. Cloning and expression of chimeric antibodies are described in EP 0 120 694 A and EP 0 125 023 A.

In a further aspect the present invention provides a method of making antibodies, the method comprising employing a Misu polypeptide or a fragment thereof as an immunogen. The present invention also provides a method of screening for antibodies which are capable of specifically binding Misu polypeptide, the method comprising contacting a Misu polypeptide with one or more candidate antibodies and detecting whether binding occurs.

Preferred antibodies according to the invention are isolated, in the sense of being free from contaminants such as antibodies able to bind other polypeptides and/or free of serum components. Monoclonal antibodies are preferred for some purposes, though polyclonal antibodies are within the scope of the present invention.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The reactivities of antibodies on a sample may be determined by any appropriate means. Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. One favoured mode is by covalent linkage of each antibody with an individual fluorochrome, phosphor or laser exciting dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

Antibodies according to the present invention may be used in screening for the presence of a polypeptide, for example in a test sample containing cells or cell lysate as discussed, and may be used in purifying and/or isolating a polypeptide according to the present invention, for instance following production of the polypeptide by expression from encoding nucleic acid. Antibodies may modulate the activity of the polypeptide to which they bind and so, if that polypeptide has a deleterious effect in an individual, may be useful in a therapeutic context (which may include prophylaxis).

### Screening for Modulators of Misu

The present invention further relates to the use of a Misu polypeptide or nucleic acid molecule for screening for candidate compounds which (a) share a Misu biological activity or (b) bind to the Misu polypeptide or (c) inhibit a biological activity of a Misu polypeptide (d) inhibit the expression of Misu polypeptide.

It is well known that pharmaceutical research leading to the identification of a new drug may involve the screening of very large numbers of candidate substances, both before and even after a lead compound has been found. This is one factor which makes pharmaceutical research very expensive and time-consuming. Means for assisting in the screening process can have considerable commercial importance and utility.

By way of example, screening can be carried out to find peptidyl or non-peptidyl mimetics or inhibitors of the Misu polypeptides to develop as lead compounds in pharmaceutical research.

In this aspect of the invention, preferably the property of Misu polypeptides used to screen for modulators is its activity as a methyltransferase, and in particular as a transferase capable of methylating nucleic acid substrates. Thus, in screening assays, conveniently the activity of Misu polypeptide can be assessed by labelling a cleavable substrate which is not cleaved when methylated with a detectable label (e.g. a fluorescent or radioactive label) and measuring the amount of label released from the substrate by the action of a cutting enzyme in the presence and absence of the Misu polypeptide, e.g. by detecting the fluorescent label or in a scintillation proximity assay. Preferably, the method is for screening for modulators, and more preferably inhibitors of Misu which may be further tested for use a therapeutic, especially for the treatment of cancer.

A method of screening for a substance which modulates activity of a polypeptide may include contacting one or more test substances with a Misu polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances. Combinatorial library technology provides an efficient way of testing a potentially vast number of different substances for ability to modulate activity of a polypeptide. Such libraries and their use are known in the art. The use of peptide libraries is preferred. Prior to or as well as being screened for modulation of activity, test substances may be screened for ability to interact with the polypeptide, e.g. in a yeast two-hybrid system where the test substance is a protein (which requires that both the polypeptide and the test substance can be expressed in yeast from encoding nucleic acid). This may be used as a coarse screen prior to testing a substance for actual ability to modulate activity of the polypeptide. Alternatively, the screen could be used to screen test substances for binding to a Misu specific binding partner, to find mimetics of the Misu polypeptide, e.g. for testing as therapeutics.

In one embodiment, the present invention provides a method of identifying a compound which is capable of modulating an activity of a Misu polypeptide, the method comprising:

(a) contacting at least one candidate compound with a Misu polypeptide as defined herein under conditions in which the candidate compound and Misu polypeptide are capable of interacting;

(b) determining in an assay for a Misu activity whether the candidate compound modulates the activity; and

(c) selecting a candidate compound which modulates an activity of the Misu polypeptide.

In a preferred embodiment, the present invention provides a method of identifying a compound which is capable of inhibiting Misu polypeptide, the method comprising:

(a) contacting at least one candidate compound and a Misu polypeptide as defined herein in the presence of a substrate under conditions in which the candidate compound, Misu polypeptide and Misu substrate are capable of interacting;

(b) determining whether the candidate compound inhibits the activity of the Misu polypeptide in reacting with the substrate; and,

(c) selecting the candidate compound which inhibits the activity of the Misu polypeptide on the substrate.

Following identification of a candidate compound which modulates, or preferably inhibits, a Misu activity, the substance may be investigated further. Furthermore, it may be manufactured and/or used in preparation, i.e. manufacture or formulation, of a composition such as a medicament, pharmaceutical composition or drug.

### Diagnostic Methods

The present invention also provides the use of Misu nucleic acid or polypeptide as a diagnostic marker for cancer, e.g. by correlating this level with the amount of the Misu polypeptide, an isoform thereof, or Misu nucleic acid present in a control.

As set out below, Misu may be overexpressed in cancers including breast cancer, small cell lung carcinoma, myeloid leukaemia and lymphoma, uterine cancer and bowel cancer. Misu may also play a role in other hyperproliferative conditions, including tumour angiogenesis, chronic inflammatory disease such as rheumatoid arthritis, atherosclerosis, skin diseases such as psoriasis, eczema, and scleroderma, diabetes, diabetic retinopathy, retinopathy of prematurity and age-related macular degeneration.

In this context, there are a number of methods known in the art for analysing samples from individuals to determine the presence of Misu nucleic acid or polypeptide. The assays may determine the presence or amount of Misu nucleic acid or polypeptide in a sample from a patient, and whether the nucleic acid or polypeptide is full length or has a Misu biological activity. Examples of biological samples include blood, plasma, serum, tissue samples, tumour samples, saliva and urine. The purpose of such analysis may be used for diagnosis or prognosis, to assist a physician in determining the severity or likely course of the condition and/or to optimise treatment of it. Exemplary approaches for detecting Misu nucleic acid or polypeptides include:

(a) determining the presence or amount of Misu polypeptide in a sample from a patient, by measuring an activity of the Misu polypeptide or its presence in a binding assay; or,

(b) determining the presence of Misu nucleic acid using a probe capable of hybridising to the Misu nucleic acid;

(c) using PCR involving one or more primers based on a Misu nucleic acid sequence to determine whether the Misu transcript is present in a sample from a patient.

In one embodiment, the method comprises the steps of:

(a) contacting a sample obtained from the patient with a solid support having immobilised thereon binding agent having binding sites specific for Misu polypeptide or Misu nucleic acid;

(b) contacting the solid support with a labelled developing agent capable of binding to unoccupied binding sites, bound Misu polypeptide or nucleic acid or occupied binding sites; and,

(c) detecting the label of the developing agent specifically binding in step (b) to obtain a value representative of the presence or amount of the Misu polypeptide or nucleic acid in the sample. The binding agent preferably is a specific binding agent and has one or more binding sites capable of specifically binding to Misu or nucleic acid in preference to other molecules. Conveniently, the binding agent is immobilised on solid support, e.g. at a defined location, to make it easy to manipulate during the assay.

Examples of specific binding pairs are antigens and antibodies, molecules and receptors and complementary nucleotide sequences. The skilled person will be able to think of many other examples and they do not need to be listed here. Further, the term “specific binding pair” is also applicable where either or both of the specific binding member and the binding partner comprise a part of a larger molecule. In embodiments in which the specific binding pair are nucleic acid sequences, they will be of a length to hybridise to each other under the conditions of the assay, preferably greater than 10 nucleotides long, more preferably greater than 15 or 20 nucleotides long. There are various methods for determining the presence or absence in a test sample of a particular nucleic acid sequence, such as the sequence shown in SEQ ID NO: 1 or 3. Exemplary tests include nucleotide sequencing, hybridisation using nucleic acid immobilized on chips, molecular phenotype tests, protein truncation tests (PTT), single-strand conformation polymorphism (SSCP) tests, mismatch cleavage detection and denaturing gradient gel electrophoresis (DGGE). These techniques and their advantages and disadvantages are reviewed in Nature Biotechnology, 15:422-426, 1997.

### Therapeutic Regulation of Misu Production

The present invention also includes the use of techniques known in the art for the therapeutic down regulation of Misu expression. These include the use of antisense techniques, RNA interference (RNAi), and antibodies or small molecules that regulate Misu production. As in other aspects of the present invention, this may be used in the treatment of cancers including Burkitt's lymphoma, diffuse large B-cell lymphomas, breast cancer, prostate cancer, gastrointestinal cancer including human colon adenocarcinoma, colorectal cancer, gastric cancer, multiple myeloma, melanoma, myeloid leukaemia and lymphoma, neuroblastoma, small cell lung cancer, medullary thyroid carcinoma, retinoblastoma, alvelolar rhabdomyosarcoma, breast cancer, uterine cancer and bowel cancer. Misu may also play a role in other hyperproliferative conditions, including tumour angiogenesis, chronic inflammatory disease such as rheumatoid arthritis, atherosclerosis, skin diseases such as psoriasis, eczema, and scleroderma, diabetes, diabetic retinopathy, retinopathy of prematurity and age-related macular degeneration. Accordingly, also included within the scope of the invention are antisense oligonucleotide sequences based on the Misu nucleic acid sequences described herein. Antisense oligonucleotides may be designed to hybridise to the complementary sequence of nucleic acid, pre-mRNA or mature mRNA, interfering with the production of polypeptide encoded by a given DNA sequence (e.g. either native Misu polypeptide or a mutant form thereof), so that its expression is reduce or prevented altogether. In addition to the Misu coding sequence, antisense techniques can be used to target the control sequences of the Misu gene, e.g. in the 5′ flanking sequence of the Misu coding sequence, whereby the antisense oligonucleotides can interfere with Misu control sequences. The construction of antisense sequences and their use is described in Peyman and Ulman, Chemical Reviews, 90:543-584, (1990), Crooke, Ann. Rev. Pharmacol. Toxicol., 32:329-376, (1992), and Zamecnik and Stephenson, P.N.A.S, 75:280-284, (1974).

In addition to antisense methods, small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending in their origin. Both types of sequence may be used to down-regulate gene expression by binding to complimentary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

Accordingly, the present invention provides the use of these sequences for downregulating the expression of Misu.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed on John et al., PLOS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design of suitable siRNA and miRNA sequences, for example using resources such as Ambion's siRNA finder, see http://www.ambion.com/techlib/misc/siRNA_finder.html. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of SEQ ID NO: 1 or 3. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of SEQ ID NO: 1 or 3.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

In one embodiment, the vector may comprise a nucleic acid sequence according to the invention in both the sense and antisense orientation, such that when expressed as RNA the sense and antisense sections will associate to form a double stranded RNA. Preferably, the vector comprises the nucleic acid sequences of SEQ ID NO:10 and a sequence partially or fully complementary to SEQ ID NO:10; or SEQ ID NO: 6 and SEQ ID NO:7; or SEQ ID NO: 8 and SEQ ID NO:9; or a variant or fragment thereof. In another embodiment, the sense and antisense sequences are provided on different vectors. Preferably, the vector comprises the nucleic acid sequences of SEQ ID NO:10, a sequence partially or fully complementary to SEQ ID NO:10, SEQ ID NO: 6, SEQ ID NO:7, SEQ ID NO: 8 or SEQ ID NO:9; or a variant or fragment thereof. Preferably, the vector is retroviral vector pRS.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through-O-or-S—.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules which are more, or less, stable than unmodified siRNA.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′position and other than a phosphate group at the 5′position. Thus modified nucleotides may also include 2′substituted sugars such as 2′-O-methyl-; 2-O-alkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N-6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N-6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,diaminopurine, methylpsuedouracil, 1-methylguanine, 1-methylcytosine. Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498).

### Pharmaceutical Compositions

The present invention disclose the use of Misu inhibitors for formulation in pharmaceutical compositions, and especially compositions for the treatment of cancer, and more especially in breast cancer, colon cancer and skin cancer, and Crohn's disease. Other conditions in which Misu may be implicated and in which Misu inhibitors may be used therapeutically, include conditions associated with aberrant Myc activity. Aberrant Myc activity has been associated with various forms of cancer including Burkitt's lymphoma, diffuse large B-cell lymphomas, breast cancer, prostate cancer, gastrointestinal cancer including human colon adenocarcinoma, colorectal cancer, gastric cancer, multiple myeloma, melanoma, myeloid leukaemia and lymphoma, neuroblastoma, small cell lung cancer, medullary thyroid carcinoma, retinoblastoma, alvelolar rhabdomyosarcoma, breast cancer, uterine cancer and bowel cancer. Misu may also play a role in other hyperproliferative conditions, including tumour angiogenesis, chronic inflammatory disease such as rheumatoid arthritis, atherosclerosis, skin diseases such as psoriasis, eczema, and scleroderma, diabetes, diabetic retinopathy, retinopathy of prematurity and age-related macular degeneration. The compositions of the present invention may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins. A composition may be administered alone or in combination with other treatments, either simultaneously or sequentially, dependent upon the condition to be treated.

### Experimental

### Transgenic Mice and Microarray Analysis

The K14MycER transgenic mice used in this study were from founder line 2184C.1 and had 70 copies of the transgene (Arnold and Watt, 2001). MycER was activated by topical application of 4-hydroxytamoxifen (4OHT, 1 mg dissolved in 0.2 ml ethanol; 1 mg per mouse per day) to a shaved area of dorsal skin. The preparation of RNA and the Affymetrix oligonucleotide array analysis are described by Frye et al. 2003.

### DNA Clones and Constructs

The BLAST 2.2.9 (NCBI, Bethesda, Md.) and BLAST (Golden Path Genome Browser, Santa Cruz, Calif.) programs were used to search mouse and human EST databases and genome sequences. The IMAGE clone containing the cDNA encoding mouse Misu (No. 5350542) was obtained from the MRC UK HGMP Resource Centre (Cambridge) and confirmed by sequencing.

### Cell Culture, Transfection and Retroviral Infection

J2-3T3 cells were cultured in DMEM containing 10% donor calf serum. Human dermal fibroblasts (HDF) from newborn foreskin and the cell lines A431 (human epidermoid carcinoma), NIH 3T3 (mouse embryonic fibroblasts), EJ/28 (bladder carcinoma) and JAR (placental trophoblastoma) were maintained in DMEM supplemented with 10% FCS. Human immortalized sebocytes (SZ95) cultivated as described in Wrobel et al. 2003.

Primary human keratinocytes were isolated from neonatal foreskin and cultured in the presence of a feeder layer of J2-3T3 cells in FAD medium (1 part Ham's F12 medium, 3 parts DMEM, 1.8×10⁻⁴M adenine) supplemented with 10% fetal calf serum (FCS) and a cocktail of 0.5 μg/ml hydrocortisone, 5 μg/ml insulin, 10⁻¹⁰ M cholera toxin and 10 ng/ml epidermal growth factor (EGF), as described previously (Gandarillas and Watt, 1997).

Keratinocytes were infected with the following retroviral vectors: pBabe puro (empty vector), pBabeMycER and pBabe106ER (Gandarillas and Watt, 1997). In the 106ER construct amino acids 106-143 of cMyc have been deleted. Misu RNAi constructs (MisuRNAi: SEQ ID NO:10, MisuRNAi1: SEQ ID NO: 6 and SEQ ID NO: 7 and MisuRNAi3: SEQ ID NO: 8 and SEQ ID NO: 9) were inserted into the retroviral vector pRS (Brummelkamp et al. 2002). The sequence of Misu RNAi oligonucleotides was confirmed by sequencing (MisuRNAi: GAG ATC CTC TTC TAT GAT C). Keratinocytes were infected by co-culture with retroviral producer cells as described previously (Gandarillas and Watt, 1997) and used within one or two passages after infection. Activation of the steroid-inducible constructs was performed by adding 200 nm 4OHT (Sigma) to the culture medium. Transient transfection of cells RNAi constructs was performed using Genejuice (Novagen), according to the manufacturer's instructions. Stable RNAi transfectants were obtained by selecting for puromycin resistance for one week.

Keratinocytes were cultured on a dermal equivalent of dead, de-epidermised dermis (DED) from adult breast skin (Rikimaru et al., 1997). Fourth or fifth passage keratinocytes was seeded on DED and grown at the air-liquid interface for 14 days.

Alternatively, SCC15 cells (a squamous cell carcinoma line) were infected by co-culture with retroviral producer cells as described previously (Gandarillas and Watt, 1997) and used within one or two passages after infection. Transient transfection of cells RNAi constructs was performed using GeneJuice (Novagen), according to the manufacturer's instructions. Stable RNAi transfectants were obtained by selecting for puromycin resistance for one week.

SCC15 cells were cultured on a dermal equivalent of dead, de-epidermised dermis (DED) from adult breast skin (Rikimaru et al., 1997). Fourth or fifth passage SCC15 cells were seeded on DED and grown at the air-liquid interface for 14 days.

### Northern Blotting

Total RNA was extracted from mouse skin with TRI Reagent® (Helena BioScience) according to the manufacturer's instructions. Northern blotting was performed as described by Frye et al., 2003. The Misu probe was amplified by PCR using the forward primer 5′ATG GTG GTA AAC CAT GAC GCA TCC and the reverse primer 5′TTG TGA GCT GTT TCT GAG TCA CC.

### In Situ Hybridisation

The construct for Misu riboprobe synthesis was generated by subcloning a fragment corresponding to nucleotides 1-753 of mouse Misu into pGEM-Teasy (Promega). The construct was linearized with SphI to provide a template to generate an antisense probe. In situ hybridisation was carried out on sections of paraformaldehyde fixed, paraffin embedded mouse back skin from wild-type and K14MycER transgenic mice using ³⁵S-labeled riboprobes.

### Antibodies

Antibodies against the following proteins were used: c-Myc (N-262; Santa Cruz), α6 integrin (MP4F10; Anbazhagan et al., 1995), actin (AC-40, Sigma), nucleolin (3G4B2, Upstate), γ-tubulin (GTU-88, Sigma), transglutaminasel (BC1, Thacher et al. 1985), keratin 10 (Babco), and Ki67 (Novacastra), and H3.3 (Abcam). A peptide corresponding to the 17 amino acids PEGEEDASDGGRKRGQA located at the N-terminus of the mouse Misu protein sequence was conjugated to keyhole limpit hemocyanin (Perbio), and injected into rabbits (Harlan Sera-Lab Limited, Hillcrest, UK) to generate antisera (MFRY5). Two peptides, one corresponding to 19 amino acids located at the N-terminus (EF1: QRPEDAEDGAEGGGKRGEA) and the second to the C-terminus (EF2: ESAASTGQPDNDVTEGQRA) of protein, were used to generate rabbit antisera to human Misu. For some applications, antibodies were affinity purified on a column containing the immunogen coupled to Amino-Link Coupling Gel (Perbio).

### Immunostaining of Cells and Tissues

Sections of tumours derived from Inva6β4 transgenic and wild-type mice subjected to two stage chemical carcinogenesis were used (Owens et al. 2003). Sections from human tissues were obtained from the Histopathology Department of Cancer Research UK and the Dental Institute of the Kings College London/Guy's & St Thomas' NHS Trust with informed consent from the patients.

Tissue samples were either fixed overnight in neutral buffered formalin and embedded in paraffin or else frozen, unfixed, in OCT compound (Miles) on a frozen isopentane surface cooled with liquid nitrogen. 5 μm sections were used for haematoxylin and eosin staining, immunohistochemistry, and immunofluorescence.

Paraffin sections were dewaxed and the endogenous peroxidase was blocked with 0.18% hydrogen peroxide in methanol for 10 minutes. Tissue sections were rehydrated and microwaved in antigen retrieval solution (Bio Genex) for 20 minutes at 700 W and incubated in swine serum 1:25 for 15 minutes. The sections were incubated with the antibody (EF1) 1:250 for 35 minutes, washed twice in phosphate buffered saline (PBS) and incubated for 35 minutes with biotinylated swine anti rabbit (Dako) in a dilution of 1:500. After two washes with PBS, the sections were subjected to Streptavidin-Peroxidase (Dako) 1:500, washed twice with PBS, and incubated with peroxidase substrate (DAB; Sigma) for 2 minutes. The tissue sections were washed in water and counter stained in haematoxylin and eosin. Frozen tissue sections and cultivated cells were fixed with 2% paraformaldehyde for 10 minutes and, if necessary, treated for 5 minutes with 0.4% Triton X 100. After blocking with 10% FCS in PBS, sections were incubated for one hour with the antibodies diluted in 10% FCS in PBS. Secondary antibodies were conjugated with AlexaFluor 488 or 594 (Molecular Probes).

In some experiments cells were lysed with 0.4% Triton X100 for 3 minutes and treated with RNaseA (100 μg/ml) for 5, 10, 15 or 20 minutes prior to fixation in paraformaldehyde for 10 minutes. To label centrioles, cells were fixed in 2% paraformaldehyde, lysed under microtubule-stabilising conditions (Komarova et al. 1997) and incubated with antibodies without the prior blocking step. Mitochondria were stained with MitoTracker® and MitoFlou™ Mitochondrion-Selective Probes (Molecular Probes), according to the manufacturer's instructions.

Whole mounts of tail epidermal sheets were prepared and immunolabelled as described previously (Braun et al. 2003). Z-stack projections were obtained from approximately 30 optical sections of 1 to 4 μm. Stained preparations were viewed and photographed with a Zeiss 510 confocal microscope.

### Western Blotting

Keratinocytes or SCC15 cells were solubilised in RIPA Buffer containing protease inhibitor cocktail tablets (Roche) and an equal volume of 2% loading buffer (4% sodium dodecyl sulfate (SDS), 12% glycerol, 50 mM Tris, 2% 2-mercaptoethanol, 0.01% Serva Blue G, 4 M urea, pH 6.8). The proteins were resolved by SDS-polyacrylamide gel electrophoresis and transferred to PVDF membranes (NEN). Blots were incubated overnight at 4° C. with primary antibodies. Primary antibodies were visualized by incubating with anti-mouse or anti-rabbit IgG horseradish-peroxidase linked antibodies for 1 hour (Amersham Pharmacia), followed by the ECL™ detection kit (Amersham Pharmacia).

### Chromatin Immunoprecipitation (ChXP) Analysis

Primary human keratinocytes were fixed and lysates containing protein/DNA complexes were prepared using a ChIP Assay Kit (Upstate), according to the manufacturer's instructions with slight modifications. For each immunoprecipitation, 2 ml of diluted lysate was precleared by addition of 60 μl of blocked protein A beads (Upstate) for 2 hours at 4° C. with agitation. Samples were immunoprecipitated overnight at 4° C. with polyclonal antibodies specific for c-Myc (4 μg N262, Santa Cruz). Immune complexes were recovered by adding 60 μl of blocked protein A beads and incubating for 4 hours at 40-C. Beads were washed and eluted, and cross links were reversed as described in the manufacturer's instructions.

The eluted material was phenol/chloroform-extracted and ethanol-precipitated. DNA was resuspended in 20 μl of water. PCR was performed with 2 μl of DNA in a final volume of 25 μl. Amplified constructs were visualised on 1.4% agarose gels. Primer sequences for the E box containing promoter regions were as follows. Nucleolin: forward primer 5′ GAC AGA GTC ACT GAG CGC CCC GAG G; reverse primer 5′ GGA AAT GAT TTC TCC TCC CGT TAC C. Misu: forward primer 5′ GGC TGT CCG CGG AGC TCC TTG AG; reverse primer 5′ TAA AGT GGC CGG GAG CGG CTC CC.

### Methyltransferase Assay

The full length cDNA of the mouse Misu gene (IMAGE clone No. 5350542) was in vitro translated using the TNT® Coupled Reticulocyte Lysate System (Promega), according to the manufacturer's instructions. Translation of the full length protein was confirmed by incorporating [³⁵S] methionine (Amersham Bioscience) and confirming that a protein of the expected size was present following SDS-polyacrylamide gel electrophoresis and fluorography.

All plasmids used for the methyltransferase assay were in vitro translated using both 5′ and 3′ RNA polymerases. Constructs transcribed in the antisense direction were used as negative controls in each reaction. A Misu splice variant lacking the N terminal 236 amino acids (ΔMisu) was cloned into pBluescript (Stratagene) and served as an additional negative control. As positive controls for DNA methylation, we used a plasmid containing full length mouse Dnmt1 and SssI (New England Biolabs).

As substrates for DNA methyltransferase assays we used unmethylated and hemi-methylated double stranded oligonucleotides, as described by Szyf et al. (1991). tRNA and rRNA purified from E. coli (Sigma) were used as RNA substrates.

The methyltransferase assay was performed as described in Fuks et al. (2000), using ¹⁴C labelled S-adenosyl-L-methionine (Amersham Bioscience). After incubating the reaction at 37° C. for 1 hour, lysates containing oligonucleotides as substrates were subjected to RNase digestion, and reactions containing RNA as substrate were digested with DNase for 10 minutes. Unincorporated nuclides were removed using Microspin G25 columns (Amersham Bioscience), according to the manufacturer's instructions. The incorporated radioactivity was measured by liquid scintillation counting.

### Cell Proliferation Assay

Cells were seeded in 24 well plates and fresh 4OHT was added every second day. At the time points indicated the cells were washed in PBS and fixed with 1% glutaraldehyde for 30 minutes. After washing three times in PBS the plates were air dried and stored until all time points had been collected. Cells were then stained with 1% crystal violet for 30 minutes, washed with PBS, de-stained with 10% acetic acid for 10 minutes and read at a wavelength of 595 nm.

### Results

Misu Expression Correlates with Activation of Myc In Vitro and In Vivo

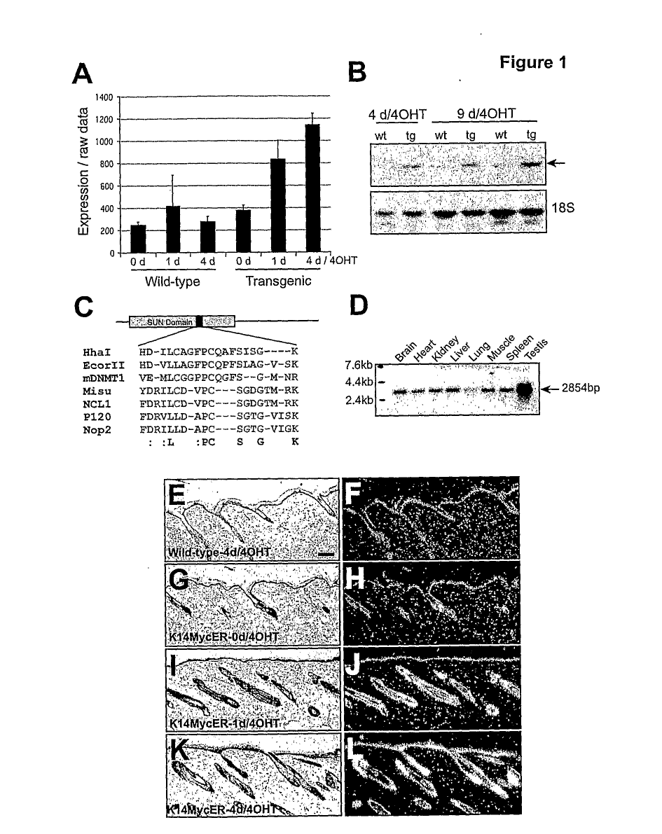

In order to analyse how Myc exerts its effect in the epidermis and to identify novel down-stream targets, microarray analysis was performed using RNA extracted from whole skin of K14MycER transgenic mice and wild-type littermates (Frye et al. 2003). K14MycER transgenic mice express human c-Myc2 fused to the G525R mutant murine estrogen binding domain (MycER) under the control of the keratin 14 promoter (Arnold and Watt, 2001). The construct is activated by topical application of 4-hydroxy-tamoxifen (4OHT). RNA was prepared from transgenic and wild-type mice that were either untreated (0 d), or treated daily for 1 day or 4 days with 4OHT. Triplicate samples, corresponding to RNA from three mice, were examined for each condition, representing a total of 18 microarrays (Frye et al., 2003; FIG. 1A).

When Myc was activated for 1 or 4 days in K14MycER epidermis there was strong up-regulation of a mRNA with the accession number BC013625 (FIG. 1A). In wild-type animals expression of BC013625 mRNA was low, whether or not the skin was treated with 4OHT (FIG. 1A). To validate the microarray results, Northern Blotting was performed using total RNA isolated from wild-type and transgenic back skin treated with 4OHT for 4 or 9 days. At both time points expression of BC013625 mRNA was elevated in K14MycER compared wild-type skin (4 d/4OHT; 9 d/4OHT; FIG. 1B).

BLAST searches with the protein sequence encoded by the mRNA revealed it to be a novel protein. A single conserved domain was detected, known as the SUN domain. SUN, also known as Fmu, was first described in E. coli and is reported to catalyze the formation of 5-methylcytidine (m⁵C) in 16S rRNA (Tscherne et al. 1999; Gu et al. 1999). The new protein was named Misu, for Myc Induced SUn domain containing protein.

The protein with the highest overall homology to Misu (36%) is yeast NCL1 (or Trm4) (Wu et al. 1998), which is a tRNA m⁵C methyltransferase (MTase; Motorin and Grosjean 1999). Misu showed weak similarity (23%) to a human protein called p120 or nucleolar associated antigen (Freeman et al. 1988). p120 is expressed in rapidly dividing cells (Freeman et al. 1991) and shows substantial homology (67%) to the yeast rRNA m⁵C MTase Nop2 (deBeus et al. 1994, King and Redman 2002).

Within the SUN domain a pair of conserved cysteines, approximately 50 amino acids apart, represent the potential active site (King and Redman 2002). FIG. 1C shows the highly conserved core sequence surrounding the first cysteine that is found in Misu, NCL1, p120 and Nop2 (Wu et al. 1998, Motorin and Grosjean 1999; FIG. 1C). This part of the SUN domain has sequence and structural similarities with motif IV of DNA:m⁵C MTases such as bacterial HhaI and EcorII and mammalian Dnmtl (Motorin and Grosjean 1999; King et al. 1999; Bujnicki et al. 2004; FIG. 1C). The conserved sequence motifs and structural homologies amongst S-adenosyl-L-methionine dependent DNA and RNA MTases strongly suggest an evolutionary relationship connecting these proteins (Reid et al. 1999, Bujnicki et al. 2004) and predict that Misu is a MTase with tRNA, rRNA or even DNA as putative substrates. In order to determine the size of the full length Misu mRNA, and to examine whether it was unique to skin or more widely expressed, tissue Northern blotting was performed (FIG. 1D). A single band of around 3 kb was detected. The published clone BC013625 is 2326 bp, but by analysing EST clones in the NCBI database we were able to predict a full length clone of 2854 bp in mouse, in good agreement with the size of the mRNA detected (FIG. 1D). The mRNA was present in all tissues analysed, with the highest expression in testis (FIG. 1D).

To examine Misu gene expression in vivo, in situ hybridisation was performed on sections of back skin from K14MycER mice and wild type littermates that were either untreated or treated daily with 4OHT for 1 day or 4 days (FIG. 1E-L). In wild-type skin treated for 4 days with 4OHT (FIGS. 1E,F) or in untreated transgenic skin (FIGS. 1G,H), Misu expression was very weak. Following 4OHT treatment of transgenic skin for 1 day (FIGS. 1I,J) or 4 days (FIGS. 1K,L) Misu expression was strongly upregulated in all cells that express the K14 promoter: the basal layer of interfollicular epidermis, the periphery of the sebaceous glands and along the entire length of the outer-root sheath of the hair follicle (Frye et al., 2003).

### Genomic Organisation and Alternative Splicing of Misu

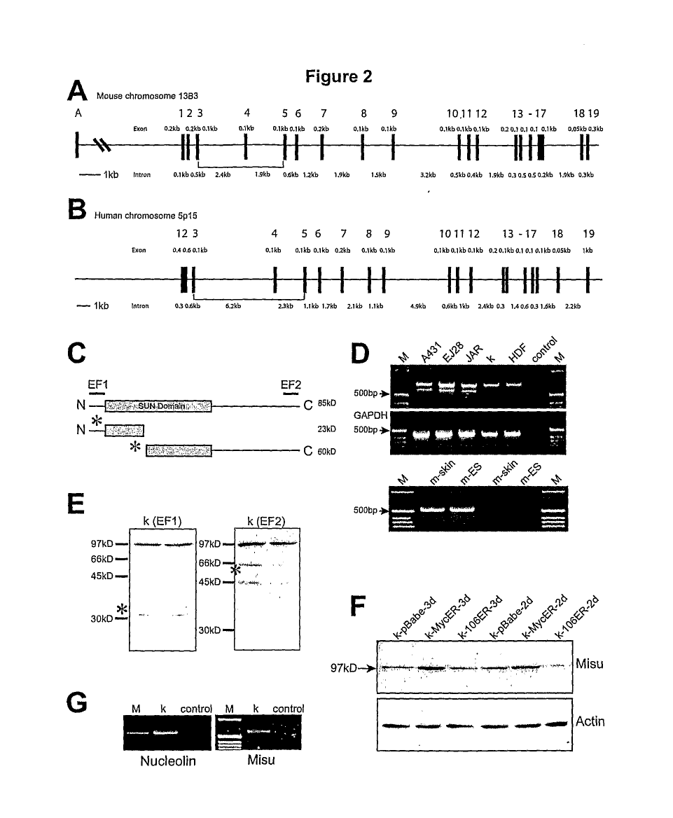

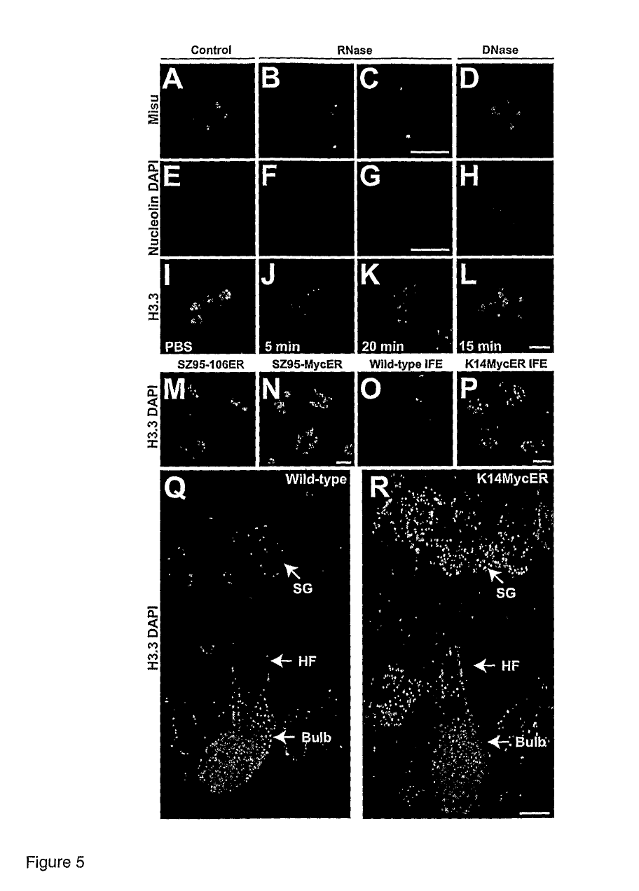

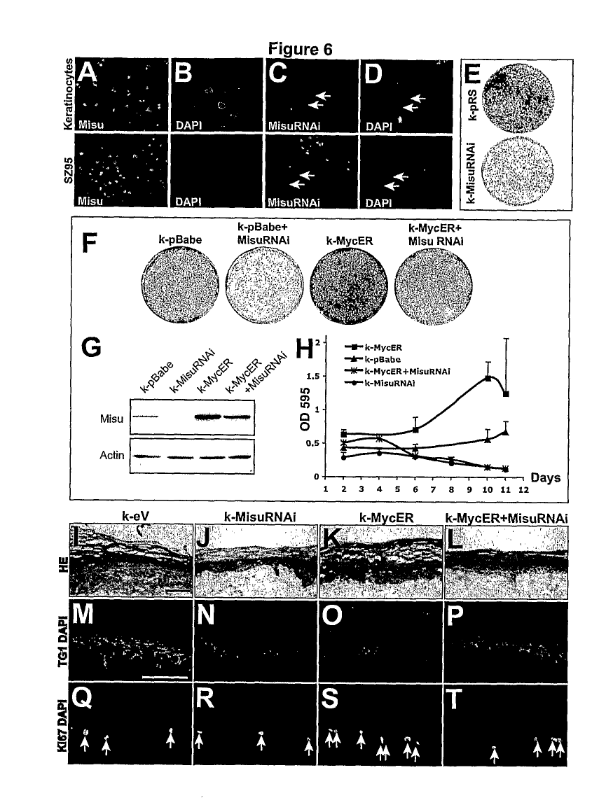

To examine the genomic organisation of Misu, the cDNA sequence was aligned with the corresponding mouse and human genomic sequences in the NCBI database. Misu localised to chromosome 13B3 in mouse (FIG. 2A) and 5p15 in human (FIG. 2B). Human and mouse Misu are each encoded by 19 exons (FIGS. 2A and B). Comparison of the full length cDNAs with EST clones suggested the existence of splice variants of Misu, two in human and three in mouse (FIG. 2A-C). The splice variant found in mouse but not human includes an additional exon (exon A) in the 5′ UTR of the mRNA, located 77 kb up-stream of the predicted start methionine, which does not affect the protein sequence (FIG. 2A). The second splice variant (common to mouse and human) results in deletion of exon 4, leading to a stop codon in exon 5, encoding a predicted protein of 23 kD (FIG. 2A-C). Shortly after the stop codon in human and mouse there is an alternative start methionine in frame with the ORF, which would encode a 60 kD protein (FIG. 2A-C). Since neither of truncated proteins would have an intact SUN domain, they might function as dominant negative inhibitors of Misu (FIG. 2C).

To determine whether any of the predicated splice variants were indeed expressed, RT-PCR was performed using RNA isolated from a range of cell types (FIG. 2D). Three human cell lines were tested, A431 (epidermoid carcinoma), EJ28 (bladder carcinoma), and JAR (placental trophoblastoma); primary human keratinocytes (k); primary dermal fibroblasts (HDF); mouse back skin (m-skin); and a mouse embryonic stem cell line (m-ES). By using one pair of primers corresponding to sequences in exons 2 and 5 a major band corresponding to the full length cDNA was detected and a 85 bp smaller fragment resulting from the deletion of exon 4 (red asterisk in FIGS. 2C, D). The smaller band was detectable in all cell types, but was always weaker than the band corresponding to full length Misu (FIG. 2D). The putative splice variant with the additional exon A could not be amplified using two other sets of primer pairs (right hand m-skin and m-ES tracks in FIG. 2D; lower panel; lane 4 and 5). Primers to look for expression of the third splice variant were not designed; however, there is evidence from Western blotting that it exists (see below).

In order to analyse whether full length Misu and the splice variants encoding the 23 and the 60 kDa proteins were translated, Western blots were performed with antibodies raised against the N- and C-terminal peptides of human Misu (FIG. 2E). Using antibody EF1 to the N-terminus, a prominent band of 97 kDa and a weaker band of approximately 30 kDa that might represent the splice variant resulting from the exon 4 deletion (FIG. 2E; red asterisk) were detected. With the C-terminal antibody EF2 the full length protein and a band at 60 kDa (FIG. 2E; blue asterisk) were detected. The additional band at 45 kDa was nonspecific (FIG. 2E and data not shown).

To analyse whether Myc activation increased Misu protein expression, Western blotting was performed of 4OHT treated cultured human keratinocytes that had been transduced with a MycER retroviral vector (k-MycER) (FIG. 2F). As controls, keratinocytes expressing the empty retroviral vector (k-pBabe) or a mutant form of MycER with a deletion within the transactivation domain (k-106ER) were also examined (FIG. 2F). Misu protein expression was increased in k-MycER after 2 or 3 days of treatment with 4OHT, but not in the control cells (FIG. 2F). There is one E box (CACGTG) located in the 5′ UTR 105 bp upstream from the predicted start methionine of human Misu. To determine whether Misu was indeed a direct target of Myc, chromatin immunoprecipitation analysis (ChIP) was performed in primary human keratinocytes (FIG. 2G). As a negative control antibodies to Myc were omitted from the lysates, and as a positive control the nucleolin promoter was used, which contains four E box elements (Greasley et al. 2000; FIG. 2G). The Misu promoter was amplified in three independent experiments, establishing that Misu is a direct transcriptional target of Myc. It is likely that the Misu signal is weak because there is only one E box and it is very close to the start methionine.

### Endogenous Misu is Nuclear and Localises to Proliferative Areas of the Skin

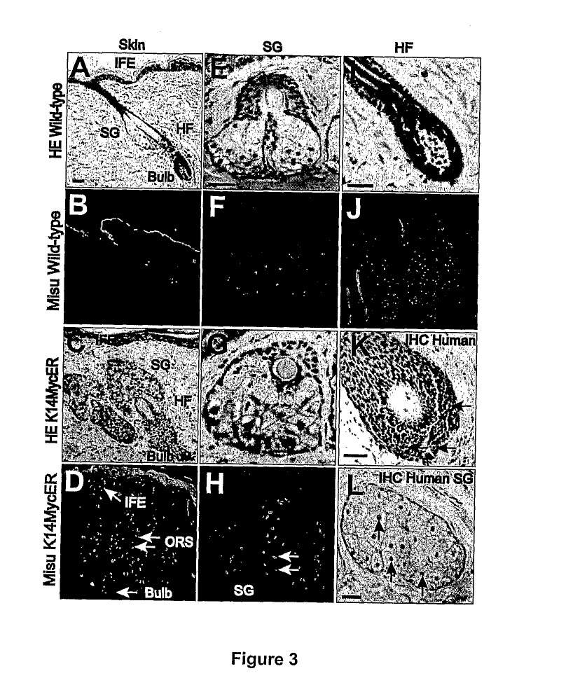

To examine the localisation of Misu protein, sections of mouse and human skin were labelled with the peptide antibodies raised against mouse or human Misu (FIG. 3A-L). In agreement with the in situ hybridisation studies (FIG. 1E-H), overall Misu expression was very weak in skin of wild-type mice (FIGS. 3A,B). However, in wild type mouse skin and in human skin strong Misu immunoreactivity was detected in cells of the sebaceous glands (FIGS. 3E,F,L) and at the base bulb (base) of growing (anagen) hair follicles (FIGS. 3I,J,K). In K14MycER mice treated with 4OHT for 4 days Misu expression was strongly increased, with immunoreactivity not only in the sebaceous glands (FIGS. 3G,H) and hair follicle bulb, but also in the basal layer of the interfollicular epidermis and along the length of the hair follicle outer root sheath (FIG. 3D), all the sites in which the transgene is expressed. In all Misu expressing cells the protein had a nuclear localisation (FIG. 3).

### Misu Methylates Nucleic Acid Targets

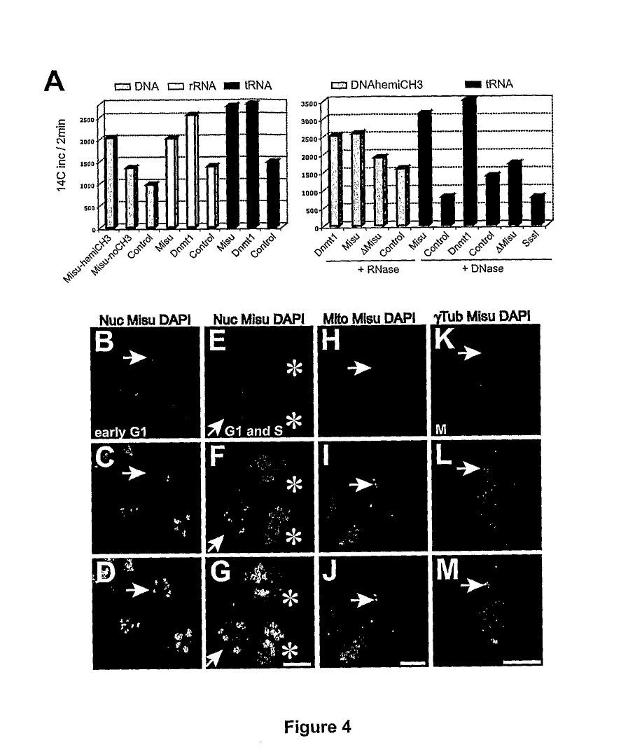

To determine whether Misu had methyltransferase activity, and to identify its target substrate, methylation assays were performed. We measured the incorporation of ¹⁴C labelled S-adenosyl-L-methionine (SAM) into RNA and DNA (FIG. 4A). Full length Misu cDNA was in vitro translated and incubated with non- (noCH3) or hemi- (hemiCH3) methylated DNA, tRNA and rRNA. Dnmt1, a DNA MTase (Bestor et al. 1988), served as a positive control. As negative controls we used Misu or Dnmt1 cDNA transcribed in the antisense direction. FIG. 4A shows the results of individual assays that were representative of the data from five independent experiments.

An approximately two fold increase in ¹⁴C incorporation into hemi-methylated DNA, tRNA and rRNA in the presence of Misu compared to controls (FIG. 4A left panel) was detected. No activity was observed on non-methylated DNA. Surprisingly, Dnmt1 methylated RNA targets as efficiently as Misu, in each case approximately two fold over the antisense controls (FIG. 4A left panel).

To exclude potential contamination of the lysates with RNA or DNA, the methylation assays were repeated, treating the RNA samples with DNase and the DNA samples with RNase (FIG. 4A, right panel). As yeast RNA:m⁵C MTases can methylate a wide range of RNAs in vitro (Obara et al. 1982) and Misu methylated both rRNA and tRNA, we used tRNA as a representative RNA target substrate. Under these conditions increased methylation activity of Misu towards tRNA (three fold) was observed compared to controls, but its ability to methylate hemi-methylated DNA was retained (FIG. 4A right panel). Dnmt1 showed the same activity towards hemi-methylated DNA and tRNA as Misu (FIG. 4A right panel). There was no significant ¹⁴C-SAM incorporation into tRNA by the 60 kD Misu splice variant (FIG. 2C; ΔMisu) or SssI, a DNA MTase isolated from Spiroplasma sp (Renbaum et al. 1990; FIG. 4A right panel).

While some yeast RNA:m⁵C MTases methylate a wide range of RNAs (e.g. tRNA, rRNA, plant virus RNA, synthetic RNA copolymers containing C) in vitro, activity towards DNA has never been detected (Obara et al. 1982; Keith et al. 1980). DNA MTases have not been tested for their ability to methylate targets other than DNA in vitro. These results confirm that Misu belongs to the m⁵C MTase protein family and demonstrate that it can accept a broad range of substrates in vitro.

### Subcellular Distribution of Misu Changes During the Cell Cycle

Although the in vitro methylation data showed that Misu was capable of methylating tRNA, rRNA and DNA, its activity in vivo must depend on its location within the nucleus. DNA MTases localise at the replication fork and their expression is increased in S-phase (Leonhardt et al.; 1992). rRNA:m⁵C MTases are found in the nucleoli (Liau and Hurlbert; 1975). tRNA MTases are found both at the nucleoli and close to the nuclear envelope, where the splicing and processing of tRNA takes place (Bertrand et al. 1998; Thompson et al. 2003). Data on the subcellular distribution of Misu obtained and validated using three different antisera to Misu, in three different cell types (mouse and human keratinocytes, and SZ95 immortalised human sebocytes) and in cells fixed with either paraformaldehyde or methanol (FIG. 4B-M).

The level of Misu varied throughout the cell cycle, with lowest expression in early G1 (FIG. 4B-D). In G1, Misu was predominantly found in the nucleoli, where it co-localised with nucleolin (arrows, FIG. 4B-D). In S phase cells the number and size of nucleoli increase and nucleolin is found in both nucleoli and nucleoplasm (Gorczyca et al. 2001; FIG. 4E, asterisks). S phase cells had the highest levels of Misu, which was distributed more uniformly throughout the nucleus (asterisks, FIGS. 4F,G) than in G1. These data were confirmed by FACS sorting S-phase cells and quantification of Misu expression by Western Blotting.

In a small percentage of cells in G2 Misu was detected in cytoplasmic vesicles (arrows, FIG. 4H-J). Although some methyltransferases are found in mitochondria (Martin and Hopper; 1994), which can be labelled with specific dyes, there was no such co-localisation of Misu (arrows in FIG. 4H-J). In M phase, Misu colocalised with γ-tubulin at the centrioles and was also found along the entire spindle (FIG. 4K-M). This distribution has never been described for RNA or DNA methyltransferases, but it has been reported that ATRX, a centromeric heterochromatin binding protein involved in methylating repetitive DNA sequences, is required for meiotic spindle organisation (De La Fuente et al. 2004).

In order to examine whether the nucleolar localisation of Misu was dependent on the presence of RNA and/or DNA, cells were treated with RNaseA or DNase prior to immunostaining (FIG. 5A-L). Five minutes of RNaseA treatment was sufficient to abolish the nucleolar localisation of Misu (FIG. 5 B). In contrast, centrioles and cytoplasmic vesicles were still positive for Misu even after 20 minutes of RNase treatment (FIG. 5C, and data not shown). DNase treatment did not effect the localisation of Misu at the nucleoli (FIG. 5D). The localisation of nucleolin was also sensitive to RNasA but not to DNase treatment (FIG. 5E-H; Schwab et al. 1998). Due to their role in ribosome biogenesis, nucleoli are highly enriched in factors involved in transcription of rRNA. The promoters of genes that are highly transcribed have chromatin modifications that enable the formation of de-condensed active euchromatin. Active chromatin was stained with an antibody to the histone variant H3.3, a chromatin modification highly enriched in rDNA genes (Ahmad and Henikoff 2002; McKittrick et al. 2004; FIG. 5I). Surprisingly, the localisation of H3.3 positive chromatin was, like Misu, dependent on RNA. After 5 minutes of RNase treatment the centre of the nucleoli were free of H3.3 chromatin and after 20 minutes H3.3 was scattered throughout the whole nucleus (FIG. 5I-K). H3.3 deposition was not affected by DNase treatment (FIG. 5L). These results show that localization of Misu and H3.3 requires nucleolar RNA species.

The role that Myc might play a role in determining and/or marking active euchromatin in the nucleoli was then investigated. MycER activation in SZ95 cells resulted in an increase of H3.3 deposition into chromatin in nucleolar structures (FIG. 5N) compared to cells expressing Myc106ER (FIG. 5M). When the back skin of MycER transgenic mice was treated with 4OHT for four days, nuclear immunoreactivity for H3.3 was increased in the IFE (FIG. 5P) compared to wild-type IFE (FIG. 5O). Using the whole mount technique to immunostain intact sheets of tail epidermis (Braun et al. 2003), in wild type epidermis we found that the cells with highest H3.3 expression were in the sebaceous glands and the bulb of anagen follicles (FIG. 5Q), the same regions that had high Misu expression (FIGS. 3F,J). On activation of Myc in K14MyER epidermis the number of cells with high H3.3 expression was greatly increased, and H3.3 expression was elevated in the sebaceous glands, IFE and hair follicles (FIG. 5R), again where Misu is upregulated (FIGS. 3D,H).

### Misu is Required for Myc Induced Proliferation

In view of the MTase activity of Misu we predicted that Misu would be required for cell proliferation. To examine this four different Misu RNAi constructs were generated and selected the one that decreased Misu protein expression most effectively in keratinocytes and SZ95, as evaluated by immunofluorescence (FIG. 6A-D) and Western blotting (FIG. 6G). When primary human keratinocytes were transduced with a Misu RNAi retroviral vector, proliferation was markedly decreased compared to the empty vector infected controls (k-pRS or k-pBabe) (FIGS. 6E,F).

Introduction of Misu RNAi into MycER transduced keratinocytes led to a decrease in Misu levels, rather than complete depletion (FIG. 6G). Nevertheless, Misu RNAi abolished the increase in growth rate of keratinocytes in response to MycER activation (FIGS. 6F,H). These data strongly suggest that Misu directly mediating Myc functions in stimulating cellular proliferation.

Myc activation in the epidermis not only stimulates proliferation (Arnold and Watt, 2001) but also promotes terminal differentiation (Gandarillas and Watt 1997; Arnold and Watt, 2001; Braun et al., 2003). To test whether this was also dependent on Misu we reconstituted human epidermis in culture by growing keratinocytes at the air-liquid interface on dead, de-epidermised dermis (DED) (Gandarillas and Watt, 1997).

Keratinocytes transduced with an empty retroviral vector (k-eV) formed distinct basal, spinous, granular, and cornified layers when cultured on DED, resembling normal epidermis (FIG. 61). As in normal human IFE, the number of proliferating cells, detected with an antibody to Ki67, was low, and they were confined to the basal layer (FIG. 6Q). Cells above the basal layer expressed a range of markers of terminal differentiation, including transglutaminase 1 and keratin 10 (FIG. 6M). Cells in the outermost, cornified, layers accumulated as anucleate squames (FIG. 6I). DEDs reconstituted with Misu deficient keratinocytes (k-MisuRNAi) showed a slight decrease in the number of cornified layers (FIG. 6 J), but there was no difference in the number or distribution of Ki67 positive cells (FIG. 6R). Terminal differentiation markers were expressed normally in the suprabasal layers, although the number of TG1 positive layers was decreased (FIG. 6N).

As reported previously (Gandarillas and Watt, 1997), keratinocytes infected with MycER formed a multilayered epidermis with disorganised suprabasal layers, more cornified layers (FIG. 6K) and increased proliferation in the basal layer (FIG. 6S). However, in the presence of Misu RNAi, these effects were abolished (FIGS. 6L,P,T) and the reconstituted epidermis resembled the control (FIGS. 6I,M,Q). We conclude that Misu RNAi not only prevents Myc induced proliferation, but also the effects of Myc on terminal differentiation.

### Misu is Highly Expressed in Mouse and Human Tumours

Following these studies on the epidermis, the hypothesis that Misu might also be expressed at low levels in other normal tissues and might be upregulated in tumours was investigated. This was tested by staining a range of normal human tissues with antibodies to Misu. Sections of human and mouse tumours were also stained.

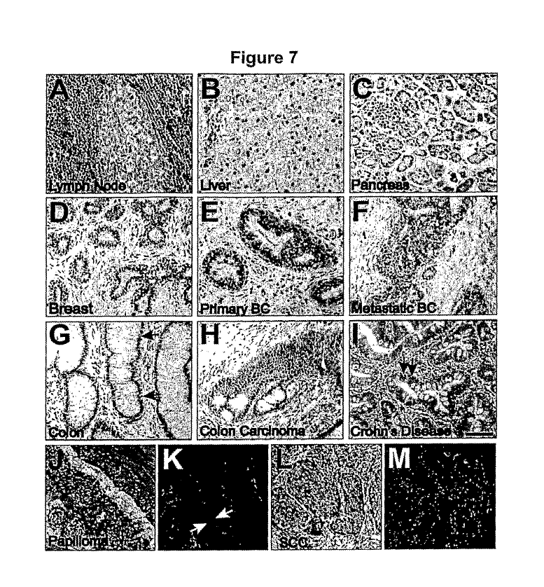

The expression of Misu was very low in a range of normal tissues (Table 1; FIGS. 7A,B and data not shown). The only exception was pancreas (FIG. 7C), where a substantial number of cells had strong nuclear immunoreactivity for Misu. In sections of normal colon Misu expression was detectable in only a small number of cell in the epithelium (FIG. 7G; arrowheads). However, in the colonic epithelium of a patient with Crohn's disease, increased expression was detected in some regions (FIG. 7I, arrowheads); this is notable because Myc is also upregulated in such tissue, and Crohn's disease predisposes to the development of gastric carcinomas (MacPherson et al. 1992). Misu expression was increased in benign (papillomas) and malignant (squamous cell carcinomas) tumours induced in mouse skin by two stage chemical carcinogenesis (FIG. 7J-M; Table 1). In papillomas, the cells expressing Misu were confined to the proliferative layers, and Misu was absent from the more differentiated cell layers (FIGS. 7J,K; arrowheads). In squamous cell carcinomas the Misu positive cells extended throughout the tumour mass (FIGS. 7L,M). Two out of five human oral squamous cell carcinomas were strongly positive for Misu (Table 1 and data not shown). Misu was upregulated in 7/7 breast carcinomas and 3/4 colon carcinomas (Table 1). Three of the breast tumours were primary tumours (FIGS. 7D,E) and four were lymph node metastases (FIG. 7F). The only tumours that did not show increased Misu expression were four rectal carcinomas (Table 1).

### Knockdown of Misu Decreases Tumour Size In Vivo

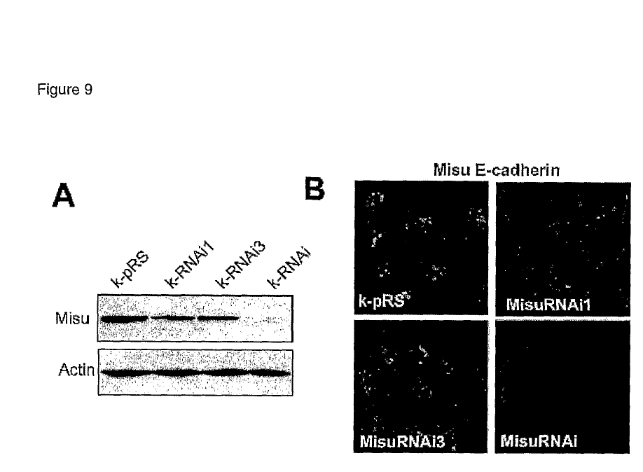

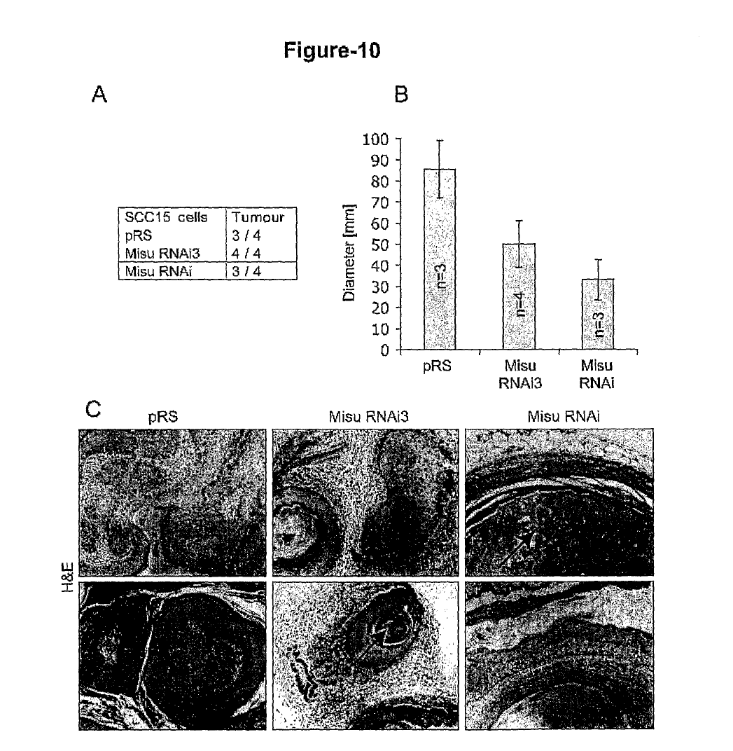

Knockdown of Misu in a squamous cell carcinoma line (SCC15) using RNAi decreased the tumour size in vivo (FIGS. 10B,C). This effect was dose-dependent (i.e. greater decrease in tumour size following greater knockdown of Misu using different RNAi's)(FIG. 9A).

### Discussion

### Defining Misu as a Myc Target Gene

Although Myc is a well known oncogene, its cellular functions in vivo, in particular in non-malignant cells are still enigmatic. Recent studies have identified a wide range of potential Myc targets and indicate that Myc binds and regulates up to 15% of all genes (Patel et al. 2004). However, it is unclear which of these targets mediate the biological functions of Myc. This work has identified and characterised a novel Myc target gene, Misu, that is up-regulated when Myc is activated in the epidermis (Arnold and Watt 2001). Misu represents a direct target gene of Myc by showing that Myc is binding to an E box located upstream of the start methionine.

The expression of Misu in the skin of K14MycER transgenic mice treated with 4OHT co-localised with activated Myc. In normal mouse and human skin, the percentage of cells that express Myc is very low confined to proliferating cells. Myc localises to the basal layer of the IFE and to keratinocytes undergoing commitment to hair shaft and inner root sheath differentiation (Rumio et al., 2000; Bull et al., 2001). Misu followed this expression pattern in normal mouse skin, it was weakly expressed in the basal layer of the IFE but up-regulated in proliferative areas such as the bulb of the anagen hair follicle.

### Misu Shows Methyltransferase Activity

BLAST searches revealed that Misu has not previously been identified in multicellular organisms. However, computer analysis identified one conserved domain, the SUN domain (Motorin and Grosjean 1999; Tscherne et al. 1999). SUN, also known as Fmu, catalyzes the formation of m⁵C of 16S rRNA (Tscherne et al. 1999; Gu et al. 1999). Fmu shows sequence homology to Trm4 (NCL1) in yeast, which is a tRNA m⁵C MTase (Wu et al. 1998; Motorin and Grosjean 1999). A conserved region of about 20 amino acids of the SUN domain is found in a number of proteins in prokaryotes, Archea, and eukaryotes described as RNA or DNA m⁵C MTases (Motorin and Grosjean 1999; King et al. 1999; Bujnicki et al. 2004). In DNA MTases this domain is part of the S-adenosyl-L-methionine-binding (AdoMet) site and the cysteine forms a covalent bond with C-6 of the pyrimidine ring during DNA methylation (Chen et al. 1991). The conserved sequence motif as well as structural homologies in AdoMet dependent DNA and RNA m⁵C MTases strongly suggest an evolutionary relationship between these two families of proteins and they might share a common ancestor (Reid et al. 1999; Buinicki et al. 2004).

The evolutionarily conserved motif within the SUN domain may explain why Misu showed methyltransferase activity towards RNA and DNA in vitro. In vivo, the nucleolar localisation of Misu was directly dependent on RNA. The tRNA:m⁵C MTase Trm4 is the protein with highest homology to Misu, and Trm4 is also localised to the nucleoli (Wu et al. 1999). This leads us to suggest that Misu is the first mammalian tRNA m⁵C MTase to be described.

Misu Mediates Myc Function in Proliferation and Regulating the Cell Cycle

The localisation and expression of Misu varied with the cell cycle, expression being highest prior to S-phase. Down-regulation of Misu in human primary keratinocytes by RNAi led to decreased proliferation. The same effect was observed in keratinocytes overexpressing Myc, suggesting that Misu directly mediates functions downstream of Myc in promoting the cell to switch from G1 to S-phase. Without wishing to be bound by any particular theory, the mechanism by which Misu regulates the cell cycle progression might be directly linked to the AdoMet synthesis pathway. L-methionine is an essential amino acid required for protein synthesis and participates together with ATP in the formation of AdoMet. On de-methylation AdoMet is converted into S-adenosyl-homocysteine (AdoHcy), which can re-enter the methyl cycle or enter the pathway to synthesize cysteine when required (for review see Fontecave et al. 2004). AdoMet is involved in regulation of G1 of the cell cycle in S. cerevisiae for example through down-regulation of G1 cyclins (Mizunuma et al. 2004). In addition, exogenous AdoMet transiently leads to G1 cell cycle delay and accumulation of AdoHcy to cell cycle arrest (Chan and Appling 2003, Christopher et al. 2002). AdoHcy has been shown to be a potent competitive inhibitor of various AdoMet-dependent MTases (Deguchi and Barchas 1971). Thus, the delay in cell cycle progression in keratinocytes expressing Misu RNAi could be explained by a temporary up-regulation of AdoMet. In transgenic mice reduced AdoMet in liver leads to increased proliferation (Lu et al. 2001). This would also explain why down-regulation of Misu in keratinocytes on DEDs had a less dramatic effect on proliferation than in cells cultured under standard growth promoting conditions. In DEDs proliferation is low and weak expression of both Myc and Misu is probably sufficient to maintain the tissue.

### Nucleolar Structures and Misu as Marker for Active Euchromatin

Most proteins localised to nucleoli are involved in ribosome biogenesis because the nucleolus is the site of synthesis and processing for rRNA, the abundant RNA involved in mRNA translation (for review see Gerbi et al. 2003). However, we believe that Misu is a member of tRNA:m⁵C MTases. Little is known about the localisation of tRNA processing in metazoan but recent studies showed in yeast that the majority of tRNA processing takes place in nucleoli (Bertrand et al. 1998; Thompson et al. 2003). This spatial juxtaposition suggests a direct coordination in three dimensions between tRNA, rRNA and mRNA (Sansam et al. 2003; Pendle et al. 2004). Thus, it is most likely that nucleolar structures represent the compartment where most if not all RNA editing takes place.

The clustering of tRNA genes in specialised subcellular regions can mark the boundaries between silenced heterochromatin and active euchromatin (Donze et al. 1999). The transcriptional potential of the tRNA gene is essential for this barrier function because it is dependent of the RNA polymerase III (Donze and Kamakaka 2001). RNA polymerase III transcription in turn is directly regulated by Myc (Felton-Edkins et al. 2003). It is possible that the large transcriptional complex functions as a physical barrier to silenced heterochromatin (Donze and Kamakaka 2001). This would fit with the concentration of the histone variant H3.3 at nucleolar structures because it marks active euchromatin (Ahmad and Henikoff 2002; McKittrick et al. 2004). H3.3 is the only histone variant that is deposited independent of DNA replication and provides a mechanism for immediate activation of genes that are silenced by histone modifications (Ahmad and Henikoff 2002). These active sites are then inherited by the daughter cells.

Activation of MycER in response to 4OHT treatment led to an increase in the amount of H3.3 positive euchromatin in the epidermal cells of transgenic mice, especially in the bulb of anagen hair follicles and the sebaceous glands. There are the major sites of Misu expression in normal skin. These observations provide a new concept of how Myc regulates cell fate and might explain the observation that in MycER transgenic mice a single application of OHT is as efficient as repeated doses in inducing the phenotype, even though the activation of c-Myc is transient (Arnold and Watt, 2001). The exact role of Myc and Misu in regulating active euchromatin by up-regulating nucleolar proteins, defining boundaries and/or influencing the deposition of H3.3 has now to be elucidated. In addition, it will be interesting to determine whether Misu and Myc play the same role in other stem cell systems.

### Myc and Misu in Malignant Transformation

Altered expression of Myc is found in a wide range of human and animal tumours, including breast carcinoma, colon carcinoma, small cell lung carcinoma, myeloid leukemias, and lymphomas (Spencer and Groudine 1991). Inappropriate Myc overexpression provides cells with a competitive advantage in growth compared to wild type cells that can lead to cancer (Donaldson and Duronio 2004).

Misu, like Myc, is up-regulated in a range of tumours. Although the mouse papillomas and squamous cell carcinomas examined have chemical induced Ras mutations (Owens et al. 2003), Myc is also known to be up-regulated in chemical induced skin tumours (Hashimoto et al. 1990).

The up-regulation of Misu in all breast carcinomas analysed was particularly striking because of the strong connection between Myc and breast cancer. A majority of breast tumours involve amplifications of Myc (Liderau et. al 1988; Liao and Dickson 2000); Myc amplification contributes to tumour progression in BRCA1-associated breast cancers (Grushko et al. 2004); and BRCA1 can directly interact with Myc (Deng and Brodie 2000). Human nucleolar associated p120 is the only known mammalian protein that showed any sequence homology to Misu and most likely represents a rRNA:m⁵C MTases family member (King et al. 1999; King and Redman 2002). Interestingly, p120 is associated with poor prognosis in breast cancer and is implicated in cell proliferation (Freeman et al. 1991; Fronagy et al. 1994; Saijo et al. 1993).

### REFERENCES

The references mentioned herein are all expressly incorporated by reference.
Adams J. M., Harris A. W., Pinkert C. A., Corcoran L. M., Alexander W. S., Cory S., Palmiter R. D., and Brinster R. L. (1985). The c-myc oncogene driven by immunoglobulin enhancers induces lymphoid malignancy in transgenic mice. Nature. 318, 533-8.
Ahmad K. and Henikoff S. (2002). The histone variant H3.3 marks active chromatin by replication-independent nucleosome assembly. Mol. Cell. 9, 1191-200.
Anbazhagan, R., Bartkova, J., Stamp, G., Pignatelli, M., Gusterson, B., and Bartek, J. (1995). Expression of integrin subunits in the human infant breast correlates with morphogenesis and differentiation. J. Pathol. 176, 227-32.
Arnold I. and Watt F. M. (2001). c-Myc activation in transgenic mouse epidermis results in mobilization of stem cells and differentiation of their progeny. Curr Biol. 11, 558-68.
Bertrand E., Houser-Scott F., Kendall A., Singer R. H., and Engelke D. R. (1998). Nucleolar localization of early tRNA processing. Genes Dev. 12, 2463-8.
Bestor T., Laudano A., Mattaliano R., and Ingram V. (1988). Cloning and sequencing of a cDNA encoding DNA methyltransferase of mouse cells. The carboxyl-terminal domain of the mammalian enzymes is related to bacterial restriction methyltransferases. J Mol. Biol. 203, 971-83.
Blackwell T. K., Huang J., Ma A., Kretzner L., Alt F. W., Eisenman R. N., and Weintraub H. (1993). Binding of myc proteins to canonical and noncanonical DNA sequences. Mol. Cell. Biol. 13, 5216-24.
Blackwood E. M. and Eisenman R. N. (1991). Max: a helix-loop-helix zipper protein that forms a sequence-specific DNA-binding complex with Myc. Science. 251, 1211-7.
Blackwood (a) E. M., Kretzner L., and Eisenman R N. (1992). Myc and Max function as a nucleoprotein complex. Curr. Opin. Genet. Dev. 2, 227-35.
Blackwood (b) E. M., Luscher B., and Eisenman R. N. (1992). Myc and Max associate in vivo. Genes Dev. 6, 71-80.
Braun K. M, Niemann C., Jensen U. B., Sundberg J. P., Silva-Vargas V., and Watt F. M. (2003). Manipulation of stem cell proliferation and lineage commitment: visualisation of label-retaining cells in wholemounts of mouse epidermis. Development. 130, 5241-55.
Brummelkamp T. R. Bernards R., and Agami R. (2002). Stable suppression of tumorigenicity by virus-mediated RNA interference. Cancer Cell. 2, 243-7.
Bujnicki J. M., Feder M., Ayres C. L., and Redman K. L. (2004). Sequence-structure-function studies of tRNA:m5C methyltransferase Trm4p and its relationship to DNA:m5C and RNA:m5U methyltransferases. Nucleic Acids Res. 32, 2453-63.
Bull, J. J., Muller-Rover, S., Patel, S. V., Chronnell, C. M., McKay, I. A., and Philpott, M. P. (2001). Contrasting localization of c-Myc with other Myc superfamily transcription factors in the human hair follicle and during the hair growth cycle. J. Invest. Dermatol. 116, 617-622.
Chan S. Y., and Appling D. R. (2003). Regulation of S-adenosylmethionine levels in Saccharomyces cerevisiae. J Biol. Chem. 278, 43051-9.
Chen L., MacMillan A. M., Chang W., Ezaz-Nikpay K., Lane W. S., Verdine and G. L. (1991). Direct identification of the active-site nucleophile in a DNA (cytosine-5)-methyltransferase. Biochemistry. 30, 11018-25.
Christopher S. A., Melnyk S., James S. J., and Kruger W. D. (2002). S-adenosylhomocysteine, but not homocysteine, is toxic to yeast lacking cystathionine beta-synthase. Mol Genet Metab. 75, 335-43.
Dalla-Favera R., Bregni M., Erikson J., Patterson D., Gallo R. C., and Croce C. M. (1982). Human c-myc one gene is located on the region of chromosome 8 that is translocated in Burkitt lymphoma cells. Proc Natl Acad Sci USA. 79, 7824-7.
de la Cova C., Abril M., Bellosta P., Gallant P., and Johnston L. A. (2004). Drosophila myc regulates organ size by inducing cell competition. Cell. 117, 107-16.
De La Fuente R., Viveiros M. M., Wigglesworth K., and Eppig J. J. (2004). ATRX, a member of the SNF2 family of helicase/ATPases, is required for chromosome alignment and meiotic spindle organization in metaphase II stage mouse oocytes. Dev Biol. 272, 1-14.
de Beus E., Brockenbrough J. S., Hong B., and Aris J. P. (1994). Yeast NOP2 encodes an essential nucleolar protein with homology to a human proliferation marker. J. Cell Biol. 127, 1799-813.
Deguchi T. and Barchas J. (1971). Inhibition of transmethylations of biogenic amines by S-adenosylhomocysteine. Enhancement of transmethylation by adenosylhomocysteinase. J Biol. Chem. 246, 3175-81.
Deng C. X. and Brodie S. G. (2000). Roles of BRCA1 and its interacting proteins. Bioessays. 22, 728-37.
Donaldson T. D. and Duronio R. J. (2004). Cancer cell biology: Myc wins the competition. Curr Biol. 14, R425-7.
Donze D. and Kamakaka R. T. (2001). RNA polymerase III and RNA polymerase II promoter complexes are heterochromatin barriers in Saccharomyces cerevisiae. EMBO J. 20, 520-31.
Donze D., Adams C. R., Rine J., and Kamakaka R. T. (1999). The boundaries of the silenced HMR domain in Saccharomyces cerevisiae. Genes Dev. 13:698-708.
Eisenman R. N. (2001). Deconstructing myc. Genes Dev. 15, 2023-30.
Felton-Edkins Z. A., Kenneth N. S., Brown T. R., Daly N. L., Gomez-Roman N., Grandori C., Eisenman R. N., and White R. J. (2003) Direct regulation of RNA polymerase III transcription by RB, p53 and c-Myc. Cell Cycle. 2, 181-4.
Fontecave M., Atta M., and Mulliez E. (2004). S-adenosylmethionine: nothing goes to waste. Trends Biochem Sci. 29, 243-9.
Frank S. R., Schroeder M., Fernandez P., Taubert S., and Amati B. (2001). Binding of c-Myc to chromatin mediates mitogen-induced acetylation of histone H4 and gene activation. Genes Dev. 15, 2069-82.
Freeman J. W., Hazlewood J. E., Auerbach P., and Busch H. (1988). Optimal loading of scraped HeLa cells with monoclonal antibodies to the proliferation-associated Mr 120,000 nucleolar antigen. Cancer Res. 48, 5246-50.
Freeman J. W., McGrath P., Bondada V., Selliah N., Ownby H., Maloney T., Busch R. K., and Busch H. (1991). Prognostic significance of proliferation associated nucleolar antigen P120 in human breast carcinoma. Cancer Res. 51, 1973-8.
Fonagy A., Swiderski C., Dunn M., and Freeman J. W. (1992). Antisense-mediated specific inhibition of P120 protein expression prevents G1- to S-phase transition. Cancer Res. 52, 5250-6.
Frye M., Gardner C., Li E. R., Arnold I., and Watt F. M. (2003). Evidence that Myc activation depletes the epidermal stem cell compartment by modulating adhesive interactions with the local microenvironment. Development. 130, 2793-808.
Fuks F., Burgers W. A., Brehm A., Hughes-Davies L., and Kouzarides T. (2000). DNA methyltransferase Dnmt1 associates with histone deacetylase activity. Nat. Genet. 24, 88-91.
Gandarillas A. and Watt F. M. (1997). c-Myc promotes differentiation of human epidermal stem cells. Genes Dev. 11, 2869-82.
Gerbi S. A., Borovjagin A. V., Ezrokhi M., and Lange T. S. (2001). Ribosome biogenesis: role of small nucleolar RNA in maturation of eukaryotic rRNA. Cold Spring Harb Symp Quant Biol. 66, 575-90.
Gorczyca W., Smolewski P., Grabarek J., Ardelt B., Ita M., Melamed M. R., and Darzynkiewicz Z. (2001). Morphometry of nucleoli and expression of nucleolin analyzed by laser scanning cytometry in mitogenically stimulated lymphocytes. Cytometry. 45, 206-13.
Greasley P. J., Bonnard C., and Amati B. (2000). Myc induces the nucleolin and BN51 genes: possible implications in ribosome biogenesis. Nucleic Acids Res. 28, 446-53.
Grushko T. A., Dignam J. J., Das S., Blackwood A. M., Perou C. M., Ridderstrale K. K., Anderson K. N., Wei M. J., Adams A. J., Hagos F. G., Sveen L., Lynch H. T., Weber B. L., and Olopade O I. (2004). MYC is amplified in BRCA1-associated breast cancers. Clin Cancer Res. 10, 499-507.
Gu X. R., Gustafsson C., Ku J., Yu M., and Santi D. V. (1999). Identification of the 16S rRNA m5C967 methyltransferase from Escherichia coli. Biochemistry. 38, 4053-7.
Hashimoto Y., Tajima O., Hashiba H., Nose K., and Kuroki T. (1990). Elevated expression of secondary, but not early, responding genes to phorbol ester tumor promoters in papillomas and carcinomas of mouse skin. Mol. Carcinog. 3:302-8.
Keith J. M., Winters E. M., and Moss B. (1980). Purification and characterization of a HeLa cell transfer RNA(cytosine-5-)-methyltransferase. J Biol Chem. 255, 4636-44.
King M. Y. and Redman K. L. (2002). RNA methyltransferases utilize two cysteine residues in the formation of 5-methylcytosine. Biochemistry. 41, 11218-25.
King M., Ton D., and Redman K. L. (1999). A conserved motif in the yeast nucleolar protein Nop2p contains an essential cysteine residue. Biochem J. 337, 29-35.
Land H., Parada L. F., and Weinberg R. A. (1983). Tumorigenic conversion of primary embryo fibroblasts requires at least two cooperating oncogenes. Nature. 304, 596-602.
Leonhardt H., Page A. W., Weier H. U., anf Bestor T. H. (1992). A targeting sequence directs DNA methyltransferase to sites of DNA replication in mammalian nuclei. Cell. 71, 865-73.
Lidereau R., Mathieu-Mahul D., Escot C., Theillet C., Champeme M. H., Cole S., Mauchauffe M., Ali I., Amione J., Callahan R., et al. (1988). Genetic variability of proto-oncogenes for breast cancer risk and prognosis. Biochimie. 70, 951-9.
Liao D. J. and Dickson R. B. (2000). c-Myc in breast cancer. Endocr Relat Cancer. 7, 143-64.
Liau M. C. and Hurlbert R. B. (1975). Interrelationships between synthesis and methylation of ribosomal RNA in isolated Novikoff Tumor nucleoli. Biochemistry. 14, 127-34.
Lu S. C., Alvarez L., Huang Z. Z., Chen L., An W., Corrales F. J., Avila M. A., Kanel G., and Mato J. M. (2001). Methionine adenosyltransferase 1A knockout mice are predisposed to liver injury and exhibit increased expression of genes involved in proliferation. Proc Natl Acad Sci USA. 98, 5560-5.
Macpherson A. J., Chester K. A., Robson L., Bjarnason I., Malcolm A. D., and Peters T. J. (1992). Increased expression of c-myc proto-oncogene in biopsies of ulcerative colitis and Crohn's colitis. Gut. 33, 651-6.
Martin N. C. and Hopper A. K. (1994). How single genes provide tRNA processing enzymes to mitochondria, nuclei and the cytosol. Biochimie. 76, 1161-7.
McKittrick E., Gafken P. R., Ahmad K., and Henikoff S. (2004). Histone H3.3 is enriched in covalent modifications associated with active chromatin. Proc Natl Acad Sci USA. 101, 1525-30.
Mizunuma M., Miyamura K., Hirata D., Yokoyama H., and Miyakawa T. (2004). Involvement of S-adenosylmethionine in G1 cell-cycle regulation in Saccharomyces cerevisiae. Proc Natl Acad Sci USA. 101, 6086-91.
Moreno E. and Basler K. (2004). dMyc transforms cells into super-competitors. Cell. 117, 117-29.
Motorin Y. and Grosjean H. (1999). Multisite-specific tRNA:m5C-methyltransferase (Trm4) in yeast Saccharomyces cerevisiae: identification of the gene and substrate specificity of the enzyme. RNA. 5, 1105-18.
Nesbit C. E., Tersak J. M., and Prochownik E. V. (1999). MYC oncogenes and human neoplastic disease. Oncogene. 18, 3004-16.
Obara M., Higashi K., and Kuchino Y. (1982). Isolation of nucleolar methylase producing only 5-methylcytidine in ribosomal RNA. Biochem Biophys Res Commun. 104, 241-6.
Owens D. M., Romero M. R., Gardner C., and Watt F. M. (2003). Suprabasal alpha6beta4 integrin expression in epidermis results in enhanced tumourigenesis and disruption of TGFbeta signalling. J Cell Sci. 116, 3783-91.
Patel J. H., Loboda A. P., Showe M. K., Showe L. C., and McMahon S. B. (2004). Analysis of genomic targets reveals complex functions of MYC. Nat. Rev. Cancer. 4, 562-8.
Pendle A. F., Clark G. P., Boon R., Lewandowska D., Lam Y. W., Andersen J., Mann M., Lamond A. I., Brown J. W., and Shaw P. J. (2004). Proteomic Analysis of the Arabidopsis Nucleolus Suggests Novel Nucleolar Functions. Mol Biol Cell. 4, 562-8.
Pinto D., Gregorieff A., Begthel H., and Clevers H. (2003). Canonical Wnt signals are essential for homeostasis of the intestinal epithelium. Genes Dev. 17, 1709-13.
Prendergast G. C., Lawe D., and Ziff E. B. (1991). Association of Myn, the murine homolog of max, with c-Myc stimulates methylation-sensitive DNA binding and ras cotransformation. Cell. 65, 395-407.
Reid R., Greene P. J., and Santi D. V. (1999). Exposition of a family of RNA m(5)C methyltransferases from searching genomic and proteomic sequences. Nucleic Acids Res. 27, 3138-45.
Renbaum P., Abrahamove D., Fainsod A., Wilson G. G., Rottem S., and Razin A. (Cloning, characterization, and expression in Escherichia coli of the gene coding for the CpG DNA methylase from Spiroplasma sp. strain MQ1 (M.SssI). Nucleic Acids Res. 18, 1145-52.
Rikimaru K., Moles J. P., and Watt F. M. (1997). Correlation between hyperproliferation and suprabasal integrin expression in human epidermis reconstituted in culture. Exp Dermatol. 6, 214-21.
Rumio, C., Donetti, E., Imberti, A., Barajon, I., Prosperi, E., Brivio, M. F., Boselli, A., Lavezzari, E., Veraldi, S., Bignotto, M., and Castano, P. (2000). c-Myc expression in human anagen hair follicles. Br. J. Dermatol. 142, 1092-1099.
Saijo Y., Perlaky L., Valdez B. C., Busch R. K., Henning D., Zhang W. W., and Busch H. (1993). The effect of antisense p120 construct on p120 expression and cell proliferation in human breast cancer MCF-7 cells. Cancer Lett. 68, 95-104.
Sansam C. L., Wells K. S., and Emeson R. B. (2003). Modulation of RNA editing by functional nucleolar sequestration of ADAR2. Proc Matl Acad Sci USA. 2003 100, 14018-23.
Satou, A., Taira, T., Iguchi-Ariga, S. M., and Ariga, H. (2001). A novel transrepression pathway of c-Myc. Recruitment of a transcriptional corepressor complex to c-Myc by MM-1, a c-Myc-binding protein. J. Biol. Chem. 276, 46562-7.
Schwab M. S., Gossweiler U., and Dreyer C. (1998). Subcellular distribution of distinct nucleolin subfractions recognized by two monoclonal antibodies. Exp Cell Res. 239, 226-34.
Spencer C. A. and Groudine M. (1991). Control of c-myc regulation in normal and neoplastic cells. Adv Cancer Res. 56, 1-48.
Staller P., Peukert K., Kiermaier A., Seoane J., Lukas J., Karsunky H., Moroy T., Bartek J., Massague J., Hanel F., and Eilers M. (2001). Repression of p151NK4b expression by Myc through association with Miz-1. Nat Cell Biol. 3, 392-9.
Szyf M., Bozovic V., and Tanigawa G. (1991). Growth regulation of mouse DNA methyltransferase gene expression. J Biol Chem. 266, 10027-30.
Taub R., Kirsch I., Morton C., Lenoir G., Swan D., Tronick S., Aaronson S., and Leder P. (1982). Translocation of the c-myc gene into the immunoglobulin heavy chain locus in human Burkitt lymphoma and murine plasmacytoma cells. Proc Natl Acad Sci USA. 79, 7837-41.
Thacher S. M., Coe E. L., and Rice R. H. (1985). Retinoid suppression of transglutaminase activity and envelope competence in cultured human epidermal carcinoma cells. Hydrocortisone is a potent antagonist or retinyl acetate but not retinoic acid. Differentiation. 29, 82-7.
Thompson M., Haeusler R. A., Good P. D., and Engelke D. R. (2004). Nucleolar clustering of dispersed tRNA genes. Science. 302, 1399-401.
Tscherne J. S., Nurse K., Popienick P., and Ofengand J. (1999). Purification, cloning, and characterization of the 16 S RNA m2G1207 methyltransferase from Escherichia coli. J Biol. Chem. 274, 924-9.
Van de Wetering M., Sancho E., Verweij C. et al. (2002). The beta-catenin/TCF-4 complex imposes a crypt progenitor phenotype on colorectal cancer cells. Cell 111, 241-50.
Waikel R. L., Kawachi Y., Waikel P. A., Wang X. J., and Roop D. R. (2001). Deregulated expression of c-Myc depletes epidermal stem cells. Nat. Genet. 28, 165-8.
Wanzel M., Herold S., and Eilers M. (2003). Transcriptional repression by Myc. Trends Cell Biol. 13, 146-50
Wilson A., Murphy M. J., Oskarsson T., et al. (2004). c-Myc controls the balance between hematopoietic stem cell self-renewal and differentiation. Genes Dev. 18, 2747-63.
Wrobel A., Seltmann H., Fimmel S., Muller-Decker K., Tsukada M., Bogdanoff B., Mandt N., Blume-Peytavi U., Orfanos C. E., and Zouboulis C C. (2003). Differentiation and apoptosis in human immortalized sebocytes. J Invest Dermatol. 120, 175-81.
Wu P., Brockenbrough J. S., and Paddy M. R., and Aris J P. (1998). NCL1, a novel gene for a non-essential nuclear protein in Saccharomyces cerevisiae. Gene. 220, 109-17.

1. An isolated polypeptide comprising the amino acid sequence set out in SEQ ID NO: 2 or 4.

2. An isolated polypeptide which has at least 80% amino acid sequence identity with the polypeptide in SEQ ID NO: 2 or 4, wherein the polypeptide has methyltransferase activity.

3. An isolated polypeptide which has at least 80% amino acid sequence identity with amino acids 1 to 236 or 237 to 767 of SEQ ID NO: 2 or 4, wherein the polypeptide is capable of inhibiting the methyltransferase activity of the polypeptide having the amino acid sequence set out in SEQ ID NO: 2 or 4.

4. The isolated polypeptide of claim 2, wherein the polypeptide has at least 90% amino acid sequence identity with the polypeptide in SEQ ID NO: 2 or 4.

5. An isolated polypeptide encoded by a nucleic acid sequence capable of hybridising under stringent conditions to the nucleotide sequence of SEQ ID NO: 1 or 3, or a complement thereof, wherein the polypeptide has methyltransferase activity or is capable of inhibiting the methyltransferase activity of the polypeptide of SEQ ID NO: 2 or 4.

6. An isolated polypeptide which is a variant, fragment or active portion of a polypeptide of claim 1, wherein said variant, fragment or active portion has methyltransferase activity or is capable of inhibiting the methyltransferase activity of the polypeptide of SEQ ID NO: 2 or 4.

7. The isolated polypeptide of claim 2 which is joined to a coupling partner.

8. The isolated polypeptide of claim 7, wherein the coupling partner is an effector molecule, a label, a drug, a toxin or a carrier or transport molecule.

9. A composition comprising the polypeptide of claim 2.

10. (canceled)

11. A method comprising screening candidate compounds to determine those which (a) share a Misu biological activity or (b) bind to the Misu polypeptide or (c) modulate a biological activity of a Misu polypeptide or (d) inhibit the expression of Misu polypeptide with a Misu polypeptide of claim 2, or a nucleic acid molecule encoding the polypeptide.

12. The method of claim 11, wherein the biological activity is methyltransferase activity.

13. The method of claim 12, wherein the modulation is inhibition of the activity of Misu polypeptide as a methyltransferase.

14. A method of identifying a compound which is capable of binding to a Misu polypeptide of claim 2, the method comprising:
(a) contacting at least one candidate compound with a Misu polypeptide under conditions in which the candidate compound and Misu polypeptide are capable of interacting;
(b) determining whether the candidate compound binds to the Misu polypeptide; and
(c) selecting a candidate compound which binds to the Misu polypeptide.

15. A method of identifying a compound which is capable of modulating an activity of a Misu polypeptide of claim 2, the method comprising:
(a) contacting at least one candidate compound with the Misu polypeptide under conditions in which the candidate compound and Misu polypeptide are capable of interacting;
(b) determining whether the candidate compound modulates the activity; and
(c) selecting a candidate compound which modulates an activity of the Misu polypeptide.

16. The method of claim 15, wherein the method further comprises determining whether the candidate compound binds to the Misu polypeptide.

17. The method of claim 15, wherein evidence of the ability of the candidate compound to modulate n activity of the Misu polypeptide is obtained by determining whether the candidate compound inhibits the methyltransferase activity of the Misu polypeptide.

18. The method of claim 17, wherein inhibiting the methyltransferase activity of the Misu polypeptide is determined by
(i) contacting the at least one candidate compound and the Misu polypeptide with a substrate for the Misu polypeptide and
(ii) determining whether the candidate compound inhibits the activity of the Misu polypeptide in reacting with the substrate.

19. The method of claim 18, wherein the substrate comprises a nucleic acid molecule capable of being methylated by the Misu polypeptide.

20. The method of claim 19, wherein the nucleic acid substrate comprises RNA.

21. The method of claim 19, wherein the nucleic acid substrate comprises a restriction site and methylation of the substrate by the Misu polypeptide inhibits the cleavage of the restriction site by a restriction enzyme.

22. The method of claim 19, wherein the nucleic acid substrate has a fluorescent label and is used in conjunction with a binding agent having a further fluorescent label, wherein the binding agent is capable of specifically binding to a methylation site present on the substrate when the substrate is methylated, so that methylation of the substrate is detectable in a fluorescent polarisation assay.

23. The method of claim 19, wherein the method comprises (i) treating the nucleic acid substrate with sodium bisulphite to alter the sequence of the substrate if it has been methylated and (ii) detecting changes in sequence by sequencing or by PCR using primers that hybridise with the methylated or unmethylated sequences.

24. The method of claim 18, wherein the substrate has a fluorescent label.

25. The method of claim 14, wherein the method is carried out in a cell based assay.

26. The method of claim 25, wherein the cells are keratinocytes.

27. The method of claim 14 wherein said method further comprises (d) manufacturing the compound of step (c) and/or formulating the compound of step (c) in a composition.

28. An antibody which is capable of specifically binding to a Misu polypeptide of claim 2.

29. A method comprising detecting and quantifying the presence of a Misu polypeptide or an isoform or splice variant thereof with an antibody of claim 28.

30. A method comprising purifying a Misu polypeptide with an antibody of claim 28.

31. A method comprising modulating activity of a Misu polypeptide with an antibody of claim 28.

32. A method of identifying a compound that modulates the expression of a Misu polypeptide of claim 2, or a nucleic acid molecule encoding the polypeptide, the method comprising:
(a) introducing a candidate compound into a cell expressing Misu polypeptide or mRNA;
(b) determining whether the Misu polypeptide or mRNA expression is altered in the presence of the candidate compound; and
(c) selecting a candidate compound which modulates the expression of the Misu polypeptide or mRNA.

33. A compound which modulates the expression of a Misu polypeptide of claim 2, or a nucleic acid encoding said polypeptide.

34. A compound according to claim 33 which inhibits the expression of Misu polypeptide or nucleic acid.

35. A compound according to claim 34 which is an antisense nucleic acid molecule, a siRNA molecule or a miRNA molecule.

36. A method for the diagnosis or prognosis of cancer or Crohn's disease, the method comprising determining the presence or amount of a Misu polypeptide of of claim 1, or an isoform or splice variant thereof, or nucleic acid encoding a Misu polypeptide, in a sample from a patient.

37. (canceled)

38. The method of claim 36, wherein the cancer is breast cancer, colon cancer, skin cancer, small cell lung carcinoma, myeloid leukaemia, lymphoma, squamous cell carcinoma or gastric carcinoma.

39. A method for the diagnosis or prognosis of cancer or Crohn's disease, wherein the method comprises determining the presence in a sample from a patient of Misu nucleic acid encoding a polypeptide of claim 2 using a probe capable of hybridising to the Misu nucleic acid; and using PCR involving one or more primers based on a Misu nucleic acid sequence to determine the presence or amount of transcript.

40. The method of claim 36, wherein the method comprises the steps of:
(a) contacting a sample obtained from the patient with a solid support having immobilised thereon binding agent having binding sites specific for Misu polypeptide or Misu nucleic acid;
(b) contacting the solid support with a labelled developing agent capable of binding to unoccupied binding sites, bound Misu polypeptide or nucleic acid or occupied binding sites; and,
(c) detecting the label of the developing agent specifically-binding in step (b) to obtain a value representative of the presence or amount of the Misu polypeptide or nucleic acid in the sample.

41. (canceled)

42. (canceled)

43. (canceled)

44. (canceled)

45. (canceled)

46. (canceled)

47. (canceled)

47. (canceled)

48. (canceled)

49. (canceled)

50. (canceled)

51. (canceled)

50. (canceled)

51-53. (canceled)

54. The method of claim 39, wherein the method comprises the steps of:
(a) contacting a sample obtained from the patient with a solid support having immobilised thereon binding agent having binding sites specific for Misu polypeptide or Misu nucleic acid;
(b) contacting the solid support with a labelled developing agent capable of binding to unoccupied binding sites, bound Misu polypeptide or nucleic acid or occupied binding sites; and,
(c) detecting the label of the developing agent specifically-binding in step (b) to obtain a value representative of the presence or amount of the Misu polypeptide or nucleic acid in the sample.

55. A method for treating a hyperproliferative condition or disease, comprising administering to a patient an effective amount of an inhibitor of a Misu polypeptide, a compound according to claim 34 or a substance which inhibits Misu expression.

56. The method of claim 55, wherein said hyperproliferative disease is cancer.

57. The method of claim 56, wherein said cancer comprises Burkitt's lymphoma, diffuse large B-cell lymphomas, prostate cancer, gastrointestinal cancer, colorectal cancer, gastric cancer, multiple myeloma, melanoma, myeloid leukaemia and lymphoma, neuroblastoma, small cell lung cancer, medullary thyroid carcinoma, retinoblastoma, alvelolar rhabdomyosarcoma, uterine cancer, breast cancer, colon cancer, skin cancer or bowel cancer.

58. The method of claim 55, wherein said hyperproliferative condition comprises Crohn's disease, tumour angiogenesis, chronic inflammatory disease, atherosclerosis, psoriasis, eczema, scleroderma, diabetes, diabetic retinopathy or retinopathy of prematurity, or age-related macular degeneration.

59. The method of claim 55, wherein said hyperproliferative condition or disease is breast cancer, colon cancer, skin cancer or Crohn's disease.

60. The method claim 55 wherein said substance is an antisense nucleic acid molecule, a siRNA molecule, a longer dsRNA molecule, a shRNA molecule or a miRNA molecule.

61. The method of claim 55 wherein the siRNA molecule, longer dsRNA molecule, shRNA molecule or miRNA molecule comprises a partial sequence of SEQ ID NO:1 or 3.

62. The method of claim 55 wherein the siRNA molecule, longer dsRNA molecule, shRNA molecule or miRNA molecule is produced endogenously by transcription from a vector.

63. The method of claim 55 wherein the siRNA molecule, longer dsRNA molecule, shRNA molecule or miRNA molecule is produced exogenously by transcription from a vector.

64. The method of claim 62 wherein said vector comprises SEQ ID NO: 6 and SEQ ID NO: 7; or SEQ ID NO: 8 and SEQ ID NO: 9; or SEQ ID NO: 10 and a sequence partially or fully complementary to SEQ ID NO: 10; or a variant or fragment thereof.

65. The method of claim 63 wherein said vector comprises SEQ ID NO: 6 and SEQ ID NO: 7; or SEQ ID NO: 8 and SEQ ID NO: 9; or SEQ ID NO: 10 and a sequence partially or fully complementary to SEQ ID NO: 10; or a variant or fragment thereof.

66. The method of claim 62 wherein said vector is retroviral vector pRS.

67. The method of claim 63 wherein said vector is retroviral vector pRS.

68. A pharmaceutical composition for the treatment of hype proliferative condition or disease comprising an effective amount of an inhibitor of a Misu polypeptide, a compound according to claim 34 or a substance which inhibits Misu expression.


SEQUENCE LISTING (19 sequences)

SEQ ID NO: 1 - DNA, 2853 bp, Mus musculus
ggatgcagccggaagtgcagcgtgcgtgcggtttggtgggtcgctgtgtgcgctccgcgtgtgcagccgcgtgggccatggggcggcgggcgcggggccggcggttccagcagccgccgcagcctgagggcgaggaagacgccagcgacggcggcagaaagcgaggccaggcgggctgggaaggtggctatcccgagatcgtaaaggagaacaagctcttcgagcactactatcaggaactcaagatcgtgccagagggagaatgggaccaattcatggagtcactccgagaacctctcccagccacactgagaatcactgggtacaaaagccatgccaaagagattctccattgcttgaagaacaagtactttaaggagttggaggacctggaagtagatggacagaaagttgaagttccacaaccactaagctggtaccctgaagaacttgcctggcatacaaacttaagtcggaaaatcttgaggaagtccccgttgttggcaaagttccatcagttcctggtcagcgagactgagtctggaaacatcagccgccaggaggctgtcagcatgatccccccactgctgctcaacgtggagccacaccataagatcttagacatgtgtgcagcccctggatccaagaccacacagttaattgaaatgttgcatgcagacatgagtgtgccctttccagagggatttgtaatcgcaaatgacgtggacaacaagcgctgctatctgctcgtccatcaggccaaaaggttgagcagtccctgcatcatggtggtaaaccatgacgcatccagcatacctagacttacagtagatgtggacggaaggaaagagattctcttctatgatcgaattttatgtgatgtcccttgcagtggcgatggcacaatgagaaaaaacattgatgtctggaagaaatggacaaccttaaacagcttgcagctccatggcctgcagcttcggattgcaactcgaggtgctgagcagctggcggaaggtggcaggatggtgtattccacgtgttccttgaaccccgtggaggatgaagcagtgatcgcagctctgctagagaagagtgaaggagctcttgagcttgctgatgtgtctgctgagttgccaggactgaagtggatgcctggagtctcacagtggaaggtcatgactagagacgggcagtggtttgcagactggcatgaggttccccagggcaggcatacacaaatccgacctaccatgttcccaccaacggacctggagaagctacaggcaatgcatctagagcgatgccttcgaatcctgccccatcatcagaatactggagggttctttgtggcagtattggtcaagaaagcaccaatgccgtggaacaaacgtcagcccaaggtccagaataaatctgcagaagccagagaacccagggtatccagccatgtggctgccacagagggaaatcccagtgaccagtctgagctggaaagtcagatgataactggagctggtgactcagaaacagctcacaacactgagaacacagagagcaatgagaagaaagatggcgtgtgtggccctcctccatcaaagaaaatgaagttgtttggatttaaagaagatccatttgtattcattcctgaagatgatcctttatttccacctattgagaagttttatgccttggatccttcattcccgaggatgaatctgttaacccgaaccacagaaggaaagaagcggcagctttatatggtctccaaggagctgaggaatgtactgctgaacaacagcgagaagatgaaggtcattaacactgggataaaagtctggtgtcgaaataacagtggtgaagaattcgattgtgcattccgtttggcacaggagggaatatatacattgtatccatttatcaattcaagaatcatcactgtatcaatggaagacgtgaagacactgttgacccaggagaacccattctttagaaaactgagcagtgaggcctacagtcaagtcaaggacctcgcaaagggaagtgttgtgctgaagtatgagccagattctgcgaatccagacaccctccagtgccccatcgtgctgtgtgggtggcggggaaaggcctctattcgaacttttgtgcccaaaaatgagcggcttcattatctcaggatgatgggtctggaggtactgggagaaaagaagaaagagggagtcattcttaccaatgagaatgctgccagcccagagcagcctggagatgaggatgccaagcagacagcacaagacccctgcgtcccagactccgtccctggctgtgatgcagctgcagctgagccatcccggtgaaggagtcttcacacaatgaattggtaccatccgttgttggagatcaaacctagaatgtccttagccagggacctggaaatgagtggtggccacagtctgttaggagtcgcttttgcagagtggatacatgtttctgctgttaaaggctaacagcctttgcagaagttcagatcccttgttggccagtattgacctagtcttctaaaacgacagtaaaagtctacaagtctttgggaagcagctctatctatctatgctttaatactttctggactgcacaccagtgtgctgtgcagtcatgctgacactgaggtctcaggtgacttctgtgcctttgtgataaagggtgagataagccatctcagagggaaggctggtgaatcatgacagcagttttggggaccctttgtgcttatcctggagttgttttaagtggggtggagattgcttgggtcatgaaataaagagctattatgatcatgaaaaaaaaaaaaaaaaaaaa

SEQ ID NO: 2 - protein, 757 aa, Mus musculus
MGRRARGRRFQQPPQPEGEEDASDGGRKRGQAGWEGGYPEIVKENKLFEHYYQELKIVPEGEWDQFMESLREPLPATLRITGYKSHAKEILHCLKNKYFKELEDLEVDGQKVEVPQPLSWYPEELAWHTNLSRKILRKSPLLAKFHQFLVSETESGNISRQEAVSMIPPLLLNVEPHHKILDMCAAPGSKTTQLIEMLHADMSVPFPEGFVIANDVDNKRCYLLVHQAKRLSSPCIMVVNHDASSIPRLTVDVDGRKEILFYDRILCDVPCSGDGTMRKNIDVWKKWTTLNSLQLHGLQLRIATRGAEQLAEGGRMVYSTCSLNPVEDEAVIAALLEKSEGALELADVSAELPGLKWMPGVSQWKVMTRDGQWFADWHEVPQGRHTQIRPTMFPPTDLEKLQAMHLERCLRILPHHQNTGGFFVAVLVKKAPMPWNKRQPKVQNKSAEAREPRVSSHVAATEGNPSDQSELESQMITGAGDSETAHNTENTESNEKKDGVCGPPPSKKMKLFGFKEDPFVFIPEDDPLFPPIEKFYALDPSFPRMNLLTRTTEGKKRQLYMVSKELRNVLLNNSEKMKVINTGIKVWCRNNSGEEFDCAFRLAQEGIYTLYPFINSRIITVSMEDVKTLLTQENPFFRKLSSEAYSQVKDLAKGSVVLKYEPDSANPDTLQCPIVLCGWRGKASIRTFVPKNERLHYLRMMGLEVLGEKKKEGVILTNENAASPEQPGDEDAKQTAQDPCVPDSVPGCDAAAAEPSR

SEQ ID NO: 3 - DNA, 2304 bp, Homo sapiens
atggggcggcggtcgcggggtcggcggctccagcaacagcagcggccggaagacgcggaggatggcgccgagggtggtggaaagcgcggcgaggcgggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaagccacgcaaaagagattctccattgcttaaagaacaaatattttaaggaattggaggacctggaggtggacggtcagaaagttgaagttccacagccactgagttggtatcctgaagaacttgcctggcacacaaatttaagtcgaaaaatcttgagaaaatcgccacacttggaaaagtttcatcagtttctagttagtgaaacagaatctggaaatattagtcgtcaagaagctgttagcatgatcccaccactgctcctcaacgtgcggcctcatcataagatcttagatatgtgtgcagcacctggctcaaagaccacacagttaattgaaatgctacatgccgacatgaatgtcccctttccagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcagtggagacggcactatgagaaaaaacattgatgtttggaaaaagtggaccaccttaaatagcttgcagctacatggcttacagctgcggattgcaacacgcggggctgaacagctggctgaaggtggaaggatggtgtattccacgtgttcactaaaccctattgaggatgaagcagtcatagcatctttactggaaaaaagtgaaggtgctttggagcttgctgatgtgtctaatgaactgccagggctgaagtggatgcctggaatcacacagtggaaggtaatgacgaaagatgggcagtggtttacagactgggacgctgttcctcacagcagacacacccagatccgacctaccatgttccctccgaaggacccagaaaagctgcaggccatgcacctggagcgatgccttaggatattaccccatcatcagaatactggagggttttttgtggcagtattggtgaaaaaatcttcaatgccgtggaataaacgtcagccaaagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtggtcctcctccatcaaagaaaatgaagttatttggatttaaagaagatccatttgtatttattcctgaagatgacccattatttccacctattgagaaattttatgctttggatccttcattcccaaggatgaatttgttaactcggactacagaagggaagaaaaggcagctctacatggtttctaaggagttgcggaatgtgctgctgaataacagtgagaagatgaaggttattaacacggggatcaaagtctggtgtagaaataacagcggtgaagagtttgactgtgctttccggctggcacaggagggaatatatacattgtatccatttattaactcaagaattattactgtatcaatggaagatgttaagatactgttgacccaggaaaatcccttttttagaaaactcagcagtgagacctacagtcaagcaaaggacctggcaaagggaagcatcgtgctgaagtatgaaccagattctgcgaatccagacgctctgcagtgtcccatcgtcttatgcggatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccggtga

SEQ ID NO: 4 - protein, 767 aa, Homo sapiens
MGRRSRGRRLQQQQRPEDAEDGAEGGGKRGEAGWEGGYPEIVKENKLFEHYYQELKIVPEGEWGQFMDALREPLPATLRITGYKSHAKEILHCLKNKYFKELEDLEVDGQKVEVPQPLSWYPEELAWHTNLSRKILRKSPHLEKFHQFLVSETESGNISRQEAVSMIPPLLLNVRPHHKILDMCAAPGSKTTQLIEMLHADMNVPFPEGFVIANDVDNKRCYLLVHQAKRLSSPCIMVVNHDASSIPRLQIDVDGRKEILFYDRILCDVPCSGDGTMRKNIDVWKKWTTLNSLQLHGLQLRIATRGAEQLAEGGRMVYSTCSLNPIEDEAVIASLLEKSEGALELADVSNELPGLKWMPGITQWKVMTKDGQWFTDWDAVPHSRHTQIRPTMFPPKDPEKLQAMHLERCLRILPHHQNTGGFFVAVLVKKSSMPWNKRQPKLQGKSAETRESTQLSPADLTEGKPTDPSKLESPSFTGTGDTEIAHATEDLENNGSKKDGVCGPPPSKKMKLFGFKEDPFVFIPEDDPLFPPIEKFYALDPSFPRMNLLTRTTEGKKRQLYMVSKELRNVLLNNSEKMKVINTGIKVWCRNNSGEEFDCAFRLAQEGIYTLYPFINSRIITVSMEDVKILLTQENPFFRKLSSETYSQAKDLAKGSIVLKYEPDSANPDALQCPIVLCGWRGKASIRTFVPKNERLHYLRMMGLEVLGEKKKEGVILTNESAASTGQPDNDVTEGQRAGEPNSPDAEEANSPDVTAGCDPAGVHPPR

SEQ ID NO: 5 - DNA, 3319 bp, Homo sapiens
aggctgtccgcggagctccttgagcccgcgtgggaaacctgggcgcttcgcgcggagacgcctttgtggctgcttcctggagcatttttttcccctagagctgttcgctgttttccctgtcacgccgcttttctgaaagacgggttgtgcgctttgggcttattcctggacggtccccaaaagtccccgaaaggaccaggcccggacacgtggggccttggtttccggccggaagtcccgggaggtgtagggctagagttctggccgtggcgggccggtttctgcgtgctgcgtgcgcggccgcgtgggctatggggcggcggtcgcggggtcggcggctccagcaacagcagcggccggaagacgcggaggatggcgccgagggtggtggaaagcgcggcgaggcgggctgggaaggaggctaccccgagatcgtcaaggagaacaagctgttcgagcactactaccaggagctcaagatcgtgcccgagggcgagtggggccagttcatggacgctctcagggagccgctcccggccactttaagaattactggttacaaaagccacgcaaaagagattctccattgcttaaagaacaaatattttaaggaattggaggacctggaggtggacggtcagaaagttgaagttccacagccactgagttggtatcctgaagaacttgcctggcacacaaatttaagtcgaaaaatcttgagaaaatcgccacacttggaaaagtttcatcagtttctagttagtgaaacagaatctggaaatattagtcgtcaagaagctgttagcatgatcccaccactgctcctcaacgtgcggcctcatcataagatcttagatatgtgtgcagcacctggctcaaagaccacacagttaattgaaatgctacatgccgacatgaatgtcccctttccagagggatttgttattgcgaatgatgtggacaacaagcgctgctacctgctcgtccatcaagccaagaggctgagcagcccctgcatcatggtggtcaaccatgatgcctccagcatacccaggctccagatagatgtggacggcaggaaagagatcctcttctatgatcgaattttatgtgatgtcccttgcagtggagacggcactatgagaaaaaacattgatgtttggaaaaagtggaccaccttaaatagcttgcagctacatggcttacagctgcggattgcaacacgcggggctgaacagctggctgaaggtggaaggatggtgtattccacgtgttcactaaaccctattgaggatgaagcagtcatagcatctttactggaaaaaagtgaaggtgctttggagcttgctgatgtgtctaatgaactgccagggctgaagtggatgcctggaatcacacagtggaaggtaatgacgaaagatgggcagtggtttacagactgggacgctgttcctcacagcagacacacccagatccgacctaccatgttccctccgaaggacccagaaaagctgcaggccatgcacctggagcgatgccttaggatattaccccatcatcagaatactggagggttttttgtggcagtattggtgaaaaaatcttcaatgccgtggaataaacgtcagccaaagcttcagggtaaatctgcagagaccagagaaagcacacagctgagccctgcagatctcacagaagggaaacccacagatccctctaagctggaaagtccgtcattcacaggaactggtgacacagaaatagctcatgcaactgaggatttagagaataatggcagtaagaaagatggcgtgtgtggtcctcctccatcaaagaaaatgaagttatttggatttaaagaagatccatttgtatttattcctgaagatgacccattatttccacctattgagaaattttatgctttggatccttcattcccaaggatgaatttgttaactcggactacagaagggaagaaaaggcagctctacatggtttctaaggagttgcggaatgtgctgctgaataacagtgagaagatgaaggttattaacacggggatcaaagtctggtgtagaaataacagcggtgaagagtttgactgtgctttccggctggcacaggagggaatatatacattgtatccatttattaactcaagaattattactgtatcaatggaagatgttaagatactgttgacccaggaaaatcccttttttagaaaactcagcagtgagacctacagtcaagcaaaggacctggcaaagggaagcatcgtgctgaagtatgaaccagattctgcgaatccagacgctctgcagtgtcccatcgtcttatgcggatggcggggaaaggcctccattcgaacttttgtgcccaagaatgaacggcttcattatctcaggatgatggggctggaggtattgggagaaaagaagaaggaaggggttatcctcacaaatgagagtgcagccagcaccggacagccagacaatgacgtgactgagggacagagagcaggagagcccaacagcccagatgcagaagaggccaacagtccagacgtgacagcaggctgtgacccggcgggggtccatccaccccggtgagcaggcccaaggcagcgggggcccacacccctcacacgcaaaactggcttcttctggtcactggtgtctgaaaccaaatccagagcagcctgtggcctgtaaagcatatatttctaatgactgcagactggtgggatcataggagccttctgaatgaccaggactgctttctttggagctgatgaaaatgtactcttttagcgtgttagaaatcacttgttttattttgtttctttggccaagctgggtctagtgtttcttttgctgggaatagactttcaaaagttgtacttctatcaagaaacaaaactgcccttgcagaaatttcaggtcttttgttaagcctgtattggtcttaaggtgcagtattttttaaattattatttatagaaagaatctataaattcttggggaagtgtgttataagctttaataattacattgagctgcacctcagtggtgtgtcattaacatgcagtggggttaatatctgaggcctcagatgactttgtgccttttggaataaagggtaaaataaactctcccagagtaagagctgtatcgtgaattgtcatactaattattgagggggacttatgtgcttttattgaatggagtgctttacaatttttatttttaaatggggttgggatccttggaatatttcaataaaattgataaaatataaaaaaaaaaaaaaaaaa

SEQ ID NO: 6 - DNA, 64 bp, Artificial Sequence
  Description of Artificial Sequence Synthetic oligonucleotide
gatccccggagaacaagctgttcgagttcaagagactcgaacagcttgttctcctttttggaaa

SEQ ID NO: 7 - DNA, 64 bp, Artificial Sequence
  Description of Artificial Sequence Synthetic oligonucleotide
agcttttccaaaaaggagaacaagctgttcgagtctcttgaactcgaacagcttgttctccggg

SEQ ID NO: 8 - DNA, 64 bp, Artificial Sequence
  Description of Artificial Sequence Synthetic oligonucleotide
gatccccccatgatgcctccagcatattcaagagatatgctggaggcatcatggtttttggaaa

SEQ ID NO: 9 - DNA, 64 bp, Artificial Sequence
  Description of Artificial Sequence Synthetic oligonucleotide
agcttttccaaaaaccatgatgcctccagcatatctcttgaatatgctggaggcatcatggggg

SEQ ID NO: 10 - DNA, 19 bp, Artificial Sequence
  Description of Artificial Sequence Synthetic oligonucleotide
gagatcctcttctatgatc

SEQ ID NO: 11 - DNA, 24 bp, Artificial Sequence
  Description of Artificial Sequence Synthetic primer
atggtggtaaaccatgacgcatcc

SEQ ID NO: 12 - DNA, 23 bp, Artificial Sequence
  Description of Artificial Sequence Synthetic primer
ttgtgagctgtttctgagtcacc

SEQ ID NO: 13 - protein, 17 aa, Mus musculus
PEGEEDASDGGRKRGQA

SEQ ID NO: 14 - protein, 19 aa, Artificial Sequence
  Description of Artificial Sequence Synthetic peptide
QRPEDAEDGAEGGGKRGEA

SEQ ID NO: 15 - protein, 19 aa, Artificial Sequence
  Description of Artificial Sequence Synthetic peptide
ESAASTGQPDNDVTEGQRA

SEQ ID NO: 16 - DNA, 25 bp, Artificial Sequence
  Description of Artificial Sequence Synthetic primer
gacagagtcactgagcgccccgagg

SEQ ID NO: 17 - DNA, 25 bp, Artificial Sequence
  Description of Artificial Sequence Synthetic primer
ggaaatgatttctcctcccgttacc

SEQ ID NO: 18 - DNA, 23 bp, Artificial Sequence
  Description of Artificial Sequence Synthetic primer
ggctgtccgcggagctccttgag

SEQ ID NO: 19 - DNA, 23 bp, Artificial Sequence
  Description of Artificial Sequence Synthetic primer
taaagtggccgggagcggctccc